SEQUENCE LISTING (15 sequences)

SEQ ID NO: 1 - protein, 338 aa, Artificial Sequence
  synthesiszed sequence
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 2 - protein, 308 aa, Artificial sequence
  synthetic sequence
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPS

SEQ ID NO: 3 - protein, 695 aa, Artificial sequence [sequence omitted]

SEQ ID NO: 4 - protein, 691 aa, Artificial sequence
  synthesized sequence
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPSGGGGSGGGGSGGGGSQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLGGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMTGHHHHHH

SEQ ID NO: 5 - protein, 117 aa, Artificial sequence
  synthesized sequence
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGT

SEQ ID NO: 6 - protein, 104 aa, Artificial sequence
  synthesized sequence
DVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKL

SEQ ID NO: 7 - protein, 6 aa, Artificial sequence
  synthesized sequence
GSGGGG

SEQ ID NO: 8 - protein, 8 aa, Artificial sequence
  synthesized sequence
GSGSGGGG

SEQ ID NO: 9 - protein, 4 aa, Artificial sequence
  synthesized sequence
GGGG

SEQ ID NO: 10 - protein, 5 aa, Artificial sequence
  synthesized sequence
GGGGS

SEQ ID NO: 11 - DNA, 20 bp, Artificial sequence
  synthesized sequence
aggcgagtcaggacattagc

SEQ ID NO: 12 - DNA, 24 bp, Artificial sequence
  synthesized sequence
ctggctagcaccaccatggtggtc

SEQ ID NO: 13 - DNA, 32 bp, Artificial sequence
  synthesized sequence
ataggatcctcacgggggtgtcgtacgggctg

SEQ ID NO: 14 - DNA, 24 bp, Artificial sequence
  synthesized sequence
ctggctagcaccaccatggtggtc

SEQ ID NO: 15 - DNA, 32 bp, Artificial sequence
  synthesized sequence
ataggatcctcacccgagcactgtcagcttgg

(19) United States
(12) Patent Application Publication
(10) Pub. No.: US 2018/0311373 A1
(43) Pub. Date: Nov. 1, 2018

(54) CONJUGATE MOLECULE

(71) Applicant: King's College Hospital NHS Foundation Trust, London (GB)

(72) Inventor: Mohammad Ibrahim, London (GB)

(21) Appl. No.: 15/965,496

(22) Filed: Apr. 27, 2018

Related U.S. Application Data
(63) Continuation of application No. 13/509,162, filed on Sep. 7, 2012, now abandoned.

(30) Foreign Application Priority Data
Nov. 11, 2009 (GB) 0919751.8

(51) Int. Cl. A61K 47/68; C07K 16/28

(52) U.S. Cl. CPC A61K 47/6849; C07K 16/28

(57) ABSTRACT

This invention relates to a therapeutic molecule capable of suppressing an immune response against an organ or tissue transplantation in a patient. In particular, the invention relates to a conjugate comprising a first portion connected to a second portion, wherein the first portion binds to an MHC Class I molecule and the second portion has HLA-G activity. This conjugate may be used as a medicament to modulate immune responses and induce immunological tolerance specific to allogenic MHC complexes.

### CROSS REFERENCE TO RELATED APPLICATIONS

This application is a continuation of U.S. patent application Ser. No. 13/509,162, filed Sep. 7, 2012, which is a 35 U.S.C. § 371 filing of International Application No. PCT/GB2010/002086, filed Nov. 11, 2010, which claims priority to United Kingdom Patent Application No. 0919751.8, filed Nov. 11, 2009, each of which are incorporated herein by reference in their entireties.

### TECHNICAL FIELD

This invention is in the field of immunomodulation. In particular, the invention relates to a therapeutic molecule capable of suppressing an immune response against an organ or tissue transplantation in a patient. The invention also relates to inducing tolerance against an organ or tissues transplantation in a patient.

### BACKGROUND ART

Transplantation is one of the most challenging and complex areas of medicine, and involves the transfer of a tissue or organ from a donor to a recipient patient. It offers the possibility to replace the recipient's damaged or defective tissue or organ with a functional one and can significantly improve the health and well-being of the recipient.

However, organ or tissue transplantation between genetically non-identical mammals is usually fraught with clinical complications which arise from immunological rejection. Immunological rejection arises due to sensitisation of the cell-mediated immune system of the recipient to the foreign (allogeneic) antigens of the donor. In particular, the recipient's immune system reacts to the major histocompatibility complex (MHC) molecules presented on the surface of the donor tissues (the graft).

The MHC molecules are expressed on the surface of cells in all jawed vertebrates, and are responsible for displaying antigens to cytotoxic T cells. The genes encoding the MHC molecules are found in the MHC region of the vertebrate genome, although the gene composition and genomic arrangement vary widely.

In humans, these genes are referred to as human leukocyte antigen (HLA) genes. The most intensely studied HLA genes are the nine so-called classical MHC genes: HLA-A, HLA-B, HLA-C, HLA-DPA1, HLA-DPB1, HLA-DQA1, HLA-DQB1, HLA-DRA, and HLA-DRB1.

In humans, the MHC is divided into three regions: Class I, II, and III. The A, B, and C genes belong to MHC class I, whereas the six D genes belong to class II.

One of the most striking features of the MHC genes, in particular in humans, is the astounding allelic diversity found therein, and especially among the nine classical genes. In humans, the most conspicuously-diverse loci, HLA-A, HLA-B, and HLA-DRB1, have roughly 250, 500, and 300 known alleles respectively. The MHC gene is the most polymorphic in the genome. It is this diversity which is responsible for immunological rejection.

The large allelic variation present in the population means that it is highly unlikely that a perfect match between all HLA molecules expressed on the allograft and the recipient's body is achieved. HLA class I and II mismatches between donor and recipient of allogeneic transplants play an important role in defining the risk of chronic graft rejection, although this is especially true for MHC Class I molecules. For example, following transplantation, recognition of non-self MHC Class I molecules by the recipient triggers an immune response which results in the cell being targeted for apoptosis. This ultimately leads to immunological rejection of the allograft. While the HLA class I molecules A, B & C are dominant, class II molecules, HLA-DR in particular, seem to be as important, especially for solid organ grafts, e.g. kidney.

Immunological rejection can usually be alleviated by administering immunosuppressant drugs to the recipient, both prior to and after the transplantation. Immunosuppressants decrease the activity of the recipient's immune system, thereby preventing it from attacking the donor tissue or organ and thus allowing better graft retention. However, the administration of immunosuppressants does not result in the patient developing long-term tolerance to the allograft, and, therefore, most patients must undergo immunosuppressive therapy for the lifetime of the graft (typically 5-10 years) or the remainder of their lives.

Immunosuppressants are known to cause a number of complications, largely owing to their non-specificity. For example, the physical side effects associated with corticosteroids such as Prednisolone, a well known immunosuppressant, include acne, striae (stretch marks), cushingoid facies (build up of fatty tissue around the face), truncal obesity, easy bruising, osteoporosis and weight gain. In addition, patients receiving immunosuppressive therapy are sensitive to opportunistic infection, which if left untreated can rapidly result in systemic infection and death.

Tailoring drug regimens to individual patients can help manage the side effects associated with immunosuppressive therapy, but it is a laborious and expensive process.

Alternative attempts have been made to overcome the limitations associated with conventional immunosuppression therapy. For example, these approaches include transient chimaerism, and co-transplantation of engineered cytotrophoblast-derived endovascular cells.

Transient chimaerism involves the allograft being transplanted together with an intravenous transfusion of bone marrow from genetically related donors, such as parents or siblings of the graft recipient. However, this approach is highly restricted because the potential bone marrow donors are limited to those with closely matched HLA haplotypes to the recipient. This approach carries significant morbidity, and can result in graft-versus-host and autoimmune disease due to the infusion of immunocompetent cells in patients who are destined to receive immunosuppression.

Co-transplantation of engineered cytotrophoblast-derived endovascular cells has also been used to provide localised immune suppression and improved graft retention. However, this approach also suffers from the limitation of only providing short term tolerance to the allograft. Additionally, the protective effect is restricted to vascular immune attack. Once immune cells extravasate from the blood vessels into the tissue, this method will afford no protection to the graft. Furthermore, this method will have local effects only at the site of the graft, and will not influence allo-sensitisation which takes place in the draining lymph nodes. Any sensitisation that occurs locally in the graft, will not take place inside the vessels, but will occur in the microenvironment of the graft itself in the extra-vascular compartment.

There is therefore a need in the art for therapeutics which overcome the disadvantages associated with conventional immunosuppressive therapy and preferably provide a means of inducing long term allograft tolerance.

### DISCLOSURE OF THE INVENTION

The inventor has surprisingly found that the mechanism which protects a foetus from the maternal immune system during pregnancy can be exploited to protect a donor graft from the recipient's immune system.

Accordingly, the invention provides a conjugate comprising a first portion connected to a second portion, wherein the first portion binds to an MHC molecule and the second portion has HLA-G activity.

By adapting aspects of the mechanisms responsible for foetal tolerance the inventor has produced a therapeutic molecule (termed the “G-body”) that can overcome the disadvantages associated with conventional immunosuppressive therapy and provide a means of improving long term graft tolerance. The conjugate of the invention is thus a fusion of two functionally active moieties to create a single molecule that has two functional ends. One binds specifically to the allogeneic HLA molecules of the donor cells in the transplanted graft, and the other displays HLA-G activity to interact with inhibitory receptors on the surface of the recipient's immune cells. In short, the conjugates of the invention mediate their effect by multiple mechanisms: i) protecting the graft from immune attack by coating its cells thus allowing the second portion of the invention (with HLA-G activity) to inhibit effector cells of the recipient such as NK, CD4 and CD8 T cells; ii) preventing sensitisation against the allo-antigens of the graft; and iii) induction of long-term tolerance by generating tolerogenic donor allogeneic DCs or tolerogenic recipient DCs that are loaded with the allo-antigens of the graft.

The most common type of allograft rejection is mediated by cellular immune mechanisms in which T cells play a central role. The same components of the immune system are involved in the alloreactivity of graft-versus-host disease (GVHD) in haematopoietic stem cell transplantation and organ transplants containing significant numbers of donor lymphoid tissue, e.g. liver transplantation. Both major types of T cells, CD4 helper cells of the TH1 type and alloreactive cytotoxic CD8 effector cells, contribute to cellular immune alloreactivity in organ rejection and GVHD. (Le Moine et al (2002) Transplantation 73:1373-1381).

There are two phases to the alloreactive immune response, the sensitisation and the effector phases. The initial event of allorecognition by T cells can take place via two important pathways. T cells can recognise allogeneic human leukocyte antigen (HLA) molecules presented on donor antigen presenting cells (APCs), known as “direct” allorecognition. In contrast, T cells can recognise donor-derived peptides presented on the HLA molecules of recipient APCs, which is referred to as “indirect” allorecognition. Both pathways of recognition are important in the sensitisation and effector phases of the alloreactive immune response.

There are multiple T cell sensitisation and effector mechanisms involved in allograft rejection and GVHD which are summarised as follows.

Cellular Movement During Allosensitisation

Donor transplanted tissues contain resident immature dendritic cells (DCs) which become activated in response to inflammatory signals initiated by tissue injury from the transplantation process itself. Consequently, these DCs, known as “passenger” leukocytes, (Hart & Fabre (1981) J. Exp. Med. 154:347-361) quickly migrate out of the graft to the secondary lymphoid tissues of the recipient and acquire characteristics of mature (activated) DCs, such as high levels of class I and II HLA molecules, as well as co-stimulatory molecules CD80 and CD86 (Larsen et al (1990) J. Exp. Med. 171:307-314). In secondary lymphoid tissues, these DCs stimulate naïve alloreactive donor T cells (direct allorecognition). Recipient naïve T cells constantly circulate between blood and secondary lymphoid tissue. When they are stimulated by a donor APC in secondary lymphoid tissue, they differentiate into effector T cells which acquire the capability of circulating through peripheral tissues including the transplanted graft. In the early phase after solid vascularised transplants, sensitisation of recipient T cells may occur in the graft itself by donor endothelial cells and DCs as lymphatic vessels take time to be re-established.

Following transplantation, donor passenger leukocytes are eventually depleted with time, thus the role of direct allorecognition diminishes. However, recipient APCs migrate into the graft in response to inflammatory cytokines and chemokines released at the site of transplantation. As these cells pass through the graft, they phagocytose debris from injured tissues and endocytose soluble antigen released by the graft. These internalised donor-derived antigens are processed by the recipient APCs and presented to naïve recipient T cells in secondary lymphoid tissues (indirect allorecognition) (Benichou et al (1992) J. Exp. Med. 175:305-308). The indirect pathway is available for presentation of donor antigens for as long as the graft persists, and does not diminish with time, although its kinetics and magnitude may be less than the direct pathway.

Direct Allorecognition in Graft Rejection and GVHD

The T cell receptors (TcRs) of both CD4 and CD8 T cells interact with allogeneic HLA molecules on the donor APCs of the class II and class I variety, respectively. Both types of T cells require co-stimulatory signals from donor APCs to become fully activated. Professional donor APCs, usually DCs, provide such signals in secondary lymphoid tissue. CD4 T cells of the TH1 type provide “help” to stimulate the proliferation, differentiation and activation of the cytotoxic alloreactive CD8 T cells. This help takes the form of secretion of cytokines, e.g. IL-2 and licensing of the APC by interferon-gamma and cross linking CD40 by CD40 ligand, so that the APCs in turn can further activate cytotoxic CD8 effector T cells, which can then damage the graft (FIG. 1). However, CD8 T cells can reject allografts and induce GVHD in the absence of CD4 helper cells, albeit with slower kinetics (FIG. 2).

Indirect Allorecognition

Considerable evidence supports the indirect pathway of CD4 T cell-mediated allorecognition in graft rejection (Jankovic et al (2002) Immunity. 16:429-439 and Fangmann et al (1992) J. Exp Med. 175:1521-1529). It plays a major role in the production of class-switched alloantibody response by allowing CD4 T cells to provide help to B cells. Numerous studies have demonstrated that the precursor frequency of T cells responding to alloantigens via the indirect pathway provides the best correlation with clinical rejection. Indirect allorecognition is considered the major pathway of chronic rejection, partly because of the importance of alloantibodies and partly because of the eventual replacement of donor APCs with recipient APCs during the natural history of a transplanted graft.

Antigens that are exogenous to the APC (such as donor-derived HLA peptides) are usually presented on class II HLA molecules to be recognised by CD4 T cells. In contrast, antigens endogenous to the APC, i.e. proteins produced in the cytosol, are usually presented on class I HLA molecule to be recognised by CD8 T cells (Liu et al (1993) J. Exp. Med. 177:1643-1650 and Neefjes & Ploegh (1992) Immunol. Today 13:179-184)). For professional APCs, especially DCs, exogenous antigen can also be presented on class I HLA molecules for recognition by CD8 T cells (Bevan (1976) J. Exp. Med. 143:1283-1288) and Sigal & Rock (2000) J. Exp. Med. 192:1143-1150). This is known as cross-priming. Indirect allorecognition by CD8 T cells involves presentation of an exogenous antigen (donor-derived HLA peptides) by recipient DCs on class I HLA molecules to be recognised by CD8 T cells, which are referred to as cross-primed CD8 T cells (FIG. 3). This pathway has been demonstrated experimentally and requires shared class I HLA alleles for effective cytotoxicity. However, even without class I sharing, cross-primed CD8 T cells can reject allografts by recognition of donor-derived alloantigens presented by recipient endothelial cells lining re-vascularised grafts, and by other indirect effector mechanisms.

A conjugate according to the invention acts by disrupting mechanisms of graft rejection at multiple points in the pathways described above. Furthermore, the conjugate harnesses one of the most important mechanisms of regulating immune responses through the induction of allo-specific regulatory T cells. Through its ability to bind avidly to MHC molecules that are specific to cells on a graft, the conjugates achieve higher concentrations at microenvironments that are rich in this allogeneic molecule. For example, in a donor-recipient combination where the donor is HLA-A2 positive and the recipient is not, the conjugates bind to the HLA-A2 on the surface of donor APCs, as well as on all parenchymal cells of the graft since class I HLA molecules (such as A2) are ubiquitously expressed on all nucleated cells. In contrast, it will not bind to the recipient cell surface, which will be devoid of HLA-A2 molecules. By binding to the MHC (in this example, HLA-A2) on the surface of donor APCS, the conjugate masks the foreign MHC and therefore interferes with direct allorecognition of this molecule, thus preventing sensitisation of CD8 T cells via the direct route. Any directly sensitised CD8 T cells that are specific to A2 cannot recognise this molecule on the surface of the graft's parenchymal cells because of the conjugate binding to their surface, which prevents direct A2-specific cytotoxicity. Direct recognition of other allogeneic HLA molecules on the surface of donor APCs coated with conjugate results in inhibition of the allogeneic CD8 T cells rather than stimulation. This will be achieved through the inhibitory properties of the HLA-G domain of the conjugate as the interaction will mimic HLA-G positive APCs, which have been shown experimentally to inhibit T cells and induce regulatory T cells (FIGS. 4 & 5) (LeMaoult (2004) Proc. Natl. Acad. Sci. U.S.A 101:7604-7609).

In one respect, the conjugates of the invention inhibit donor APCs via its HLA-G activity interacting with inhibitory leukocyte Immunoglobulin-like receptors (LILRs) (Brown et al (2004) Tissue Antigens 64:215-225) on the cell surface. Consequently, these donor APCs become tolerogenic and induce regulatory T cells in the recipient that are specific to the allogeneic HLA molecules of the donor, i.e. donor-specific T cell tolerance. In this instance, allogeneic donor HLA molecules are directly presented to the recipient's T cells (direct allorecognition, FIG. 6). The conjugates interact with donor APCs via both of its functional domains. In addition to the HLA-G/LILR interaction (see above), the conjugate binds to the donor APC via its anti-MHC properties (again, in this example, HLA-A2). This masks HLA-A2 molecules at least partially. Any unmasked HLA-A2 molecules will be presented by a tolerogenic donor APC (due to binding of HLA-G) which will induce HLA-A2-specific tolerance.

The ability to bind HLA-A2 also allows conjugates of the invention to coat dead donor cells (both APCs and parenchymal cells), as well as any particulate material containing HLA-A2 which is shed from donor tissues (cell debris). As these particles are cleared by the phagocytic cells, the HLA-G activity of the conjugate will engage the inhibitory LILRs on these innate immune cells. Therefore, donor-derived peptides including HLA-A2 and other allogeneic donor molecules will be processed and presented by conjugate-conditioned recipient APCs, which will be tolerogenic (indirect allorecognition, FIG. 7). These APCs will induce donor-specific adaptive regulatory T cells (Tr1) which would promote graft tolerance (FIG. 8). Due to the preferred absence of an Fc portion of the immunoglobulin component of the conjugate, the binding with graft MHC will not result in activation of complement or engagement of activating Fc receptors on innate immune cells. Therefore, no inflammatory mediators will be released. Similar mechanisms will operate in the case of foreign MHC-containing soluble macromolecular entities released from donor tissues. The conjugates form an inhibitory immune complex with such molecules, thus enhancing tolerogenic alloantigen presentation.

The conjugates induce similar protective mechanisms in case of GVHD.

Preferably the first portion of the conjugate of the invention comprises an antibody. Preferably the antibody is a monoclonal antibody. Preferably the antibody is an antigen binding immunoglobulin fragment. Preferably, the antigen binding immunoglobulin fragment does not include an Fc portion.

The first portion of the conjugate may comprise an aptamer.

Preferably the first portion and second portion of the conjugate are connected by a linker. Preferably the linker is a peptide.

Preferably the second portion of the conjugates of the invention comprises HLA-G or a fragment thereof. Preferably the second portion comprises the polypeptide sequence given as SEQ ID NO: 1 or SEQ ID NO:2 or is a fragment or variant thereof.

The invention also includes a pharmaceutical composition comprising a conjugate of the invention.

The invention also includes a method of preventing graft rejection in a transplant patient comprising administering a conjugate or a pharmaceutical composition of the invention to the patient before, during and/or after transplant surgery.

The invention also includes a method of inducing tolerance to a graft in a transplant patient comprising administering a conjugate or pharmaceutical composition of the invention to the patient before, during and/or after transplant surgery.

The conjugates of the invention thus have a two-fold function. Firstly, upon binding, the first portion masks the non-self MHC molecules found on the transplanted tissue or organ and prevents their recognition and attack by the host cytotoxic T-cells. Secondly, binding of the first portion brings the HLA-G activity of the second portion within close proximity to the cell surface of the transplanted tissue. The HLA-G activity of the molecule then suppresses NK cell activity towards the transplanted tissue or organ (Pazmany et al (1996) Science. 274:792-795).

When the complex formed by the non-self MHC molecules present on the transplanted tissue or organ and the conjugates of the invention are processed by the host immune system (e.g. by dendritic cells) long-term graft tolerance is induced. Tolerogenic DCs (from donor or recipient) that will be generated by the conjugates of the invention can induce and maintain the long-term tolerance by inducing T cell anergy, deleting the alloreactive T cells altogether and/or generating allo-specific regulatory T cells. As described in detail above, the inventors believe that the conjugate leads to long-term tolerance of a graft in a recipient because any host dendritic cells which interact with the graft are simultaneously exposed to HLA-G activity. These dendritic cells subsequently internalise the complex comprising the MHC molecule and HLA-G activity. In the course of acquiring this complex, the inhibitory receptors of the dendritic cells are engaged by HLA-G causing these cells to become tolerogenic to the antigen. (Horuzsko et al (2001) Int. Immunol. 13:385-394). As a result, when these dendritic cells migrate to the lymph node and encounter T cells, tolerance is induced in the T cells towards the graft antigens which are presented by these dendritic cells.

The conjugates of the invention therefore provide a mechanism by which local immunosuppression of a transplanted tissue or organ can be achieved without the need for systemically suppressing the host's immune system. Thus, the patient will not suffer from the side-effects associated with conventional immunosuppressive therapy.

In example 6, we have shown a synergistic effect of HLA-G tetramers and anti-HLA-A2 when combined in the form of G-Body. This is a novel concept. Furthermore, HLA-A2 expression by immune cells seems to be a pre-requisite for the inhibitory effect to take place as demonstrated by experiments performed with HLA-A2 positive as opposed to HLA-A2 negative donors. Thus, these data support the concept that immune responses to antigens other than HLA-A2 would not be unduly compromised and confirms that the effect of G-Body would be specific to the HLA mismatch in a transplantation setting and therefore would be unlikely to cause blanket immunosuppression. The specificity to HLA-A2 in these results supports the workings of the invention described herein.

Finally, we have demonstrated that the inhibitory effect of G-Body on Peripheral blood mononuclear cell (PBMC) proliferation is not due to any putative general toxicity of a foreign protein because there has been no significant inhibitory effect on the proliferation of HLA-A2 negative PBMCs.

Furthermore, because the conjugates of the invention are capable of inducing long term tolerance it is not necessary to treat a patient with conventional expensive and potentially harmful immunosuppression therapies for the lifetime of the transplanted tissue or organ. Tolerance, once induced, may be sustained for as long as the graft continues to be present in the recipient as this would ensure sustained supply of the alloantigen, so maximising the chances of maintaining immunological tolerance.

First Portion

As described above, the first portion of the conjugate of the invention is intended specifically to target the binding and localisation of the conjugate to an MHC molecule expressed on the surface of a graft cell. The first portion therefore has an affinity for an MHC molecule specific to the graft and must be specific to that MHC molecule, in the sense that it does not bind to MHC molecules possessed by the recipient.

As an example, we can describe a donor who is HLA-A2 positive and a recipient who is HLA-A2 negative. HLA-A2 is very common in the Caucasian population (40-50%). Thus, the approach of using a conjugate with specificity for HLA-A2 (e.g. anti-HLA-A2 antibodies) would be suitable for roughly a quarter of all transplantation cases in the Caucasian population. The first portion of the conjugate is in this example designed to be completely specific to HLA-A2 and does not cross-react with any closely related molecules in order to avoid the possibility of binding to HLA molecules of the recipient. As the skilled reader will understand, other disparate class I HLA molecules between donors and recipients can be targeted to ensure that the conjugate of the invention does not bind to recipient cells. The invention exploits the disparity in MHC signature between the donor and the recipient to ensure that the graft cells of the donor are specifically targeted. Thus, to treat grafts from a diverse population, a range of conjugates according to the invention will be required, each one specific to each of the common HLAs, e.g. HLA-A2, HLA-B8, . . . etc. For treatment, a suitable conjugate will be selected from the available range to suit each particular combination of donor and recipient. Most of the possible donor/recipient pairs will be covered by a small set of such conjugates.

Conjugates of the invention also have the potential to be used in the context of xenografts. In this case the first portion of the conjugate is specific for a unique marker of the donor species. For example, if it is a pig graft, the first portion of the invention may be an antibody that is specific to pig MHC which does not cross-react with human HLA molecules.

Preferably the first portion has a binding affinity (Kd) of less than 10⁻⁶M, i.e. 10⁻⁷M, 10⁻⁸M, 10⁻⁹M, 10⁻¹⁰M, 10⁻¹¹M, 10⁻¹²M or even 10⁻¹³M or less towards an MHC molecule. Preferably, the first portion has a binding affinity (Kd) of less than 10⁻⁹M, i.e., 10⁻¹⁰M, 10⁻¹¹M, 10⁻¹²M or 10⁻¹³M towards an MHC molecule.

The first portion may comprise one or more molecules which have an affinity for an MHC molecule.

Preferably the first portion comprises a molecule with an affinity for an MHC Class I molecule, although there is no reason why the conjugate cannot be made specific to class II HLA molecules as well. While the HLA class I molecules A, B & C are dominant, class II molecules, HLA-DR in particular, seem to be as important, especially for solid organ grafts, e.g. kidney. Accordingly, the invention embraces the notion of targeting other common HLA mismatches; both class I and class II. For example, HLA-DR or HLA-B-specific G-body conjugates will have a beneficial effect if these molecules are mismatched between donor and recipient.

MHC Class I molecules are expressed on the surface of cells in all jawed vertebrates, and are responsible for displaying antigens to cytotoxic T cells. The genes encoding the MHC molecules are found in the MHC region of the vertebrate genome, although the gene composition and genomic arrangement vary widely.

In humans, these genes are referred to as human leukocyte antigen (HLA) genes. The most intensely studied HLA genes are the nine so-called classical MHC genes: HLA-A, HLA-B, HLA-C, HLA-DPA1, HLA-DPB1, HLA-DQA1, HLA-DQB1, HLA-DRA, and HLA-DRB1. In humans, the MHC is divided into three regions: Class I, II, and III. The A, B, and C genes belong to MHC class I, whereas the six D genes belong to class II. HLA

MHC Class I protein molecules are heterodimers comprising two polypeptide chains: a highly polymorphic a chain (comprising 3 domains: α1, α2 and α3) and a non-covalently associated β2-microglobulin. Human MHC Class I protein molecules may be referred to in the art as “HLA molecules”, or “HLA protein molecules”.

Accordingly, the term “MHC Class I molecule” as used herein includes all mammalian MHC Class I molecules including human and non-human. Preferably the MHC Class I molecule is a human MHC Class I molecule (HLA protein molecule).

MHC Class I molecules are responsible for binding and presenting antigens on the cell surface and therefore exist with or without bound antigen. Accordingly, the term MHC Class I molecule refers the MHC Class I molecule either on its own, or when bound to an antigen.

The MHC Class I molecule may be an HLA molecule. Preferably the HLA molecule is a product of the HLA-A gene. The HLA-A gene is polyallelic and as such, a variety of differences in the α chain of the encoded protein exist within the population.

Preferably the HLA molecule bound by the first portion according to the invention is a product of the HLA-A2 gene.

HLA-A2 is a HLA serotype within the HLA-A ‘A’ serotype group and is encoded by the HLA-A02 allele group including the HLA-A0201, HLA-A0202, HLA-A0203, HLA-A0205, HLA-A0206, HLA-A0207 and HLA-A0211 gene products. HLA-A2 is very common in the Caucasian population (40-50%) and provides an ideal cellular target for the first portion because it will be suitable for use in many combinations of donor and recipient. This approach would be suitable to roughly a quarter of all transplantation cases in the Caucasian population. Accordingly, any member of the HLA-A2 allele group is encompassed by the term ‘HLA-A2’.

Other examples of common Class I MHC alleles targeted by the first portion of the conjugate according to the invention will be clear to the skilled reader, and for example includes HLA-B8. Common MHC Class I alleles are shown below in Table 1.

TABLE 1
Common MHC Class I Alleles

| | | | African- | | | | |
|---|---|---|---|---|---|---|---|
| Allele | Caucasian | Allele | American | Allele | Hispanic | Allele | Oriental |
| A*0201 | 45.6% | C*0401 | 29.0% | A*0201 | 37.1% | A*1101 | 38.4% |
| C*0701 | 27.7% | C*0701 | 25.4% | C*0401 | 25.4% | A*2402 | 33.7% |
| A*0101 | 27.4% | C*0602 | 23.0% | A*2402 | 24.9% | C*0702 | 33.3% |
| A*0301 | 23.8% | A*0201 | 22.3% | C*0702 | 24.2% | C*0102 | 27.7% |
| C*0702 | 21.5% | A*2301 | 20.7% | C*0701 | 20.8% | A*3303 | 23.3% |
| C*0401 | 21.2% | C*0202 | 19.0% | C*0304 | 14.4% | C*0801 | 21.6% |
| B*4402 | 20.2% | A*0301 | 18.7% | A*0301 | 14.3% | C*0304 | 19.9% |
| B*0702 | 18.1% | C*0702 | 18.1% | B*0702 | 13.2% | A*0201 | 18.1% |
| B*0801 | 18.1% | B*5301 | 18.1% | B*3501 | 12.8% | B*4001 | 15.2% |
| C*0501 | 17.2% | B*0702 | 15.8% | C*0602 | 12.3% | C*0401 | 14.0% |
| C*0304 | 16.8% | C*1601 | 15.7% | C*0501 | 11.9% | B*5801 | 13.3% |
| C*0602 | 15.7% | B*1503 | 13.9% | A*0101 | 11.4% | B*4601 | 12.7% |
| A*1101 | 15.3% | B*5801 | 13.5% | A*1101 | 11.0% | B*5101 | 12.4% |
| B*4001 | 13.6% | A*6802 | 12.7% | B*5101 | 10.8% | C*0302 | 12.0% |
| A*2402 | 12.1% | C*1701 | 11.7% | C*1601 | 10.6% | B*3802 | 11.4% |
| B*3501 | 10.7% | B*4501 | 10.8% | B*4403 | 9.9% | A*0207 | 11.0% |
| C*0303 | 10.6% | B*4201 | 10.5% | C*0102 | 9.7% | B*1501 | 9.4% |
| B*5101 | 10.4% | A*3001 | 10.4% | A*2902 | 9.7% | A*0206 | 9.3% |

The first portion of the conjugate may comprise an antibody. Antibodies may be of any isotype (e.g. IgA, IgG, IgM i.e. an α, γ or μ heavy chain). Antibodies may have a κ or λ, light chain. Within the IgG isotype, antibodies may be IgG1, IgG2, IgG3 or IgG4 subclass. The term “antibody” includes any suitable natural or artificial immunoglobulin or derivative thereof. In general, the antibody will comprise a Fv region which possesses specific antigen binding activity. This includes, but is not limited to: whole immunoglobulins, antigen binding immunoglobulin fragments (e.g. Fv, Fab, F(ab′)2 etc.), single chain antibodies (e.g. scFv), oligobodies, chimeric antibodies, humanized antibodies, veneered antibodies, etc.

Antibodies used in the conjugates of the invention may be polyclonal or monoclonal.

It is preferred that antibodies and antibody components present in the conjugates of the invention do not contain an Fc portion. This means that binding to graft MHC will not result in activation of complement or engagement of activating Fc receptors on innate immune cells. Therefore, no inflammatory mediators will be released.

Antibodies used in the conjugates of the invention may be produced by any suitable means e.g. by recombinant expression, or by administering (e.g. injecting) a polypeptide of the invention to an appropriate animal (e.g. a rabbit, hamster, mouse or other rodent).

To increase compatibility with the human immune system, the antibodies may be chimeric or humanized {e.g. Breedveld (2000) Lancet 355(9205):735-740 or Gorman & Clark (1990) Semin. Immunol. 2:457-466}, or fully human antibodies may be used. Because humanized antibodies are far less immunogenic in humans than the original non human monoclonal antibodies, they can be used for the treatment of humans with far less risk of anaphylaxis.

Humanized antibodies may be achieved by a variety of methods including, for example: (1) grafting non-human complementarity determining regions (CDRs) onto a human framework and constant region (“humanizing”), with the optional transfer of one or more framework residues from the non human antibody; (2) transplanting entire non-human variable domains, but “cloaking” them with a human-like surface by replacement of surface residues (“veneering”). As described herein, humanized antibodies will include both “humanized” and “veneered” antibodies. (Jones et al. (1986) Nature 321:522-52580, Morrison et al. (1984) Proc. Natl. Acad. Sci, US.A., 81:6851-6855, Morrison & Oi (1988) Adv. Immunol., 44:65-92, Verhoeyer et al. (1988) Science 239:1534-1536, Padlan (1991) Molec. Immun 28:489-498, Padlan (1994) Molec. Immunol. 31(3):169-217 and Kettleborough et al. (1991) Protein Eng. 4(7):773-83). CDRs are amino acid sequences which together define the binding affinity and specificity of a Fv region of a native immunoglobulin binding site (Chothia et al. (1987) J. Mol. Biol. 196:901-917.87 and Kabat et al. U.S. Dept. of Health and Human Services NIH Publication No. 91-3242 (1991)).

Humanized or fully human antibodies can also be produced using transgenic animals that are engineered to contain human immunoglobulin loci. For example, WO98/24893 discloses transgenic animals having a human Ig locus wherein the animals do not produce functional endogenous immunoglobulins due to the inactivation of endogenous heavy and light chain loci. WO91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO96/30498 discloses the use of the Cre/Lox system to modify the immunoglobulin locus in a mammal, such as to replace all or a portion of the constant or variable region to form a modified antibody molecule. WO94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a desired MHC Class I molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO96/33735. The monoclonal antibodies can be tested for the ability to inhibit or neutralize the biological activity or physiological effect of the corresponding polypeptide.

Where the first portion comprises one or more molecules it may comprise different classes of the antibodies referred to above. For example, it may comprise an Fv fragment and a Fab fragment, both of which have an affinity to an MHC Class I molecule. B11 is an example of an ScFv fragment generated using phage display technology and a process of “light-chain shuffling”, which is specific for human HLA-A2. The process is described by Watkins et al., 2004 (Molecular studies of anti-HLA-A2 using light-chain shuffling: a structural model for HLA antibody binding. Tissue Antigens. 2004 April; 63(4):345-54). As used herein, the B11 ScFv was derived from a plasmid containing the ScFv fragment obtained from the above authors; the sequence was confirmed and then modified to generate a nucleic acid molecule encoding a conjugate according to the invention. The CDR sequences for B11 are published by Watkins et al., 2000 (The isolation and characterisation of human monoclonal HLA-A2 antibodies from an immune V gene phage display library. Tissue Antigens. 2000 March; 55(3):219-28); and Watkins et al., 2004 (loc cit.) and provide examples of suitable sequences for use in a conjugate according to the present invention.

VH
QVQLVQSGGGVVQPGGSLRVSCAASGVTLSDYGMHWVRQAPGKGLEW
MAFIRNDGSDKYYADSVKGRFTISRDNSKKTVSLQMSSLRAEDTAVY
YCAKNGESGPLDYWYFDLWGRGT
VL
DVVMTQSPSSLSASVGDRVTITCQASQDISNYLNWYQQKPGKAPKLL
IYDASNLETGVPSRFSGSGSGTDFTFTISSLQPEDIATYYCQQYDNL
PPTFGGGTKL

Underlined Areas Denote the CDRs.

The first portion may comprise the same class of antibody. For example, the first portion may be a diabody, composed of two scFv fragments which are linked together by a linker peptide such as serine and/or glycine or by a chemical linker.

Antibodies against human HLA-A2 to be used in the conjugates of the invention may, for example, be derived from the hybridoma BB7.2. The BB7.2 hybridoma was obtained from the ATCC (American Tissue Culture Collection). The original publication describing this was in 1981 (Parham P, Brodsky F M. Partial purification and some properties of BB7.2. A cytotoxic monoclonal antibody with specificity for HLA-A2 and a variant of HLA-A28. Hum Immunol. 1981 December; 3(4):277-99) and the hybridoma is now commercially available. Other anti-HLA-A2 hybridomas exist and are commercially available.

The first portion may in an alternative embodiment, comprise an aptamer. By ‘aptamer’ we mean a nucleic acid sequence which, owing to said nucleic acid sequence and the environment in which this sequence is located, forms a three-dimensional structure. The three-dimensional structure confers, in part, binding properties to aptamers which have specificity to a target molecule or molecules. In the present invention, said aptamer shows binding specificity towards an MHC molecule.

Aptamers are typically oligonucleotides which may be single stranded oligodeoxynucleotides, oligoribonucleotides, or modified oligodeoxyribonucleotides or modified oligoribonucleotides. A screening method to identify aptamers is described in U.S. Pat. No. 5,270,163. The term ‘modified’ in the context of aptamers refers to nucleotides with a covalently modified base and/or sugar or sugar derivatives.

Second Portion

As described above, the second portion of the conjugate of the invention is intended to have HLA-G activity. HLA-G is a member of the non-classical human leukocyte antigen (HLA) class Ib molecules. The HLA class Ib antigens, HLA-E, —F and -G, share some characteristics with the class Ia antigens, but also differ from them in a range of ways. A comprehensive review of the functions of the HLA class Ib antigens can be found in Hviid T. 2006 (Human Reproduction Update, Vol. 12, No. 3 pp. 209-232).

HLA-G molecules form one aspect through which the foetus is protected from immunologically-induced harm from the mother. A foetus, which is for the most part the mating product of two histoincompatible individuals, is essentially an allograft. It is composed of 50% paternal genes and, therefore, produces paternal antigens that should be targeted for destruction by the maternal immune system due to recognition of them as foreign or non-self (Mellor, 2000).

Transplantation of paternal tissues or organs into the maternal environment would, unless the immune system was suppressed, elicit a cascade of immune responses resulting in the rapid clearance of the transplant from the maternal body. In the vast majority of pregnancies, however, the foetal allograft is not rejected. In fact, the maternal immune system appears to be specifically suppressed in relation to the foetus, but still able to function to protect the mother from a wide range of external pathogens.

The two aspects of the mechanism by which a foetus is tolerated by the maternal immune system which are relevant for the present invention are the adaptation of the outer layer of the placenta, called the trophoblast, and the presence of a unique HLA molecule called HLA-G.

It is known that the placenta, which is foetal-derived, sequesters the foetus away from the maternal immune system. The outermost layer of the placenta, called the trophoblast, has several adaptations which appear to underpin its resistance to the maternal immune system. One adaptation is that trophoblast cells do not display MHC class I or class II molecules on their surface. Obviously if an MHC molecule is not presented, then it cannot be recognised as non-self and therefore a cell lacking MHC class I molecules should avoid destruction by the immune system.

However, cells which do not display MHC class I molecules are vulnerable to attack by natural killer (NK) cells. NK cells are a type of cytotoxic lymphocyte that constitute a major component of the innate immune system. They are capable of specifically recognising and killing cells which do not express MHC Class I molecules.

It is thought that the cells of the trophoblast are protected from attack by NK cells by expressing and displaying on their surface an MHC molecule called human leukocyte antigen-G (HLA-G) (Human Reproduction Update, Thomas Vauvert F. Hviid (2006)). HLA-G is a specialised antigen expressed only by these cells (Hunt, 2000b). It exhibits greatly decreased polymorphism when compared to other HLA (Le Bouteiller, 2003) and specifically binds natural killer cells via their killer-cell immunoglobulin-like receptors thus blocking their cytotoxity (Furman, 2000).

Moreover, the immunomodulatory activity of HLA-G is thought to result partly from engaging inhibitory receptors of dendritic cells and down regulating CD8⁺ and CD4⁺ T-cell reactivity.

Of particular interest to the present invention is HLA-G's ability to inhibit NK and T-cell-mediated cell lysis, both through direct interaction with the receptors ILT2 and ILT4 and with the killer Ig-like receptor 2 DL4 (KIR2DL4 receptor) (Navarro et al., 1999, Eur J Immunol 29, 277-283; Ponte et al., 1999, Proc Natl Acad Sci USA 96, 5674-5679; Rajagopalan and Long, 1999, J Exp Med 189, 1093-1100; Riteau et al., 2001, Int Immunol 13, 193-201; Menier et al., 2002, Int J Cancer 100, 63-70).

Therefore, the term “HLA-G activity” refers to an ability to inhibit NK and T-cell-mediated cell lysis and/or modulate the function of myelomonocytic cells, including dendritic cells. A molecule with HLA-G activity has an ability to bind to at least one of the receptors for HLA-G, i.e. ILT2, ILT4 and KIR2DL4, or a selection of those. HLA-G binds to ILT2 and ILT4 which are expressed predominantly on myeloid immune cells (DCs and monocyte/macrophages) and to a lesser extent on lymphocytes. HLA-G also binds to KIR2DL4 on NK cells. All these receptors of HLA-G are inhibitory receptors that regulate the function of cells that express them when they are engaged with their HLA-G ligand.

The second portion may comprise one or more molecules which have HLA-G activity.

The molecule with HLA-G activity may be a polypeptide. Similarly, the molecule may be an aptamer which binds to one or more of the receptors for HLA-G, i.e. ILT2, ILT4 and KIR2DL4.

The second portion may comprise an HLA-G polypeptide. HLA-G is a heterodimer consisting of a heavy chain and a light chain (beta-2 microglobulin). The heavy chain is anchored in the membrane. The heavy chain is approximately 45 kDa and its gene contains 8 exons. Exon 1 encodes the leader peptide, exons 2 and 3 encode the alpha1 and alpha2 domain, which both are both capable of binding and displaying a peptide, exon 4 encodes the alpha3 domain, exon 5 encodes the transmembrane region, and exon 6, 7 and 8 encode the cytoplasmic tail.

HLA-G polypeptide potentially exists in four membrane-bound isoforms, HLA-G1 to -G4, and three soluble isoforms, HLA-G5 to -G7 (Ishitani and Geraghty, 1992; Fujii et al., 1994; Kirszenbaum et al., 1994; Hviid et al., 1998; Paul et al., 2000; LeMaoult et al., 2003). The soluble HLA-G isoforms are generated by alternative splicing of the HLA-G transcript. The external part of the HLA-G molecule consists of three parts: the 1, 2 and 3 domains (exons 2-4); domains 1 and 2 contribute to the peptide-binding cleft. The HLA-G full-length membrane protein is anchored in the cell membrane by the transmembrane region (exon 5).

The second portion may comprise any one of the HLA-G polypeptide variants described above.

The second portion may also comprise a fragment of one of the HLA class I antigen polypeptide fragments described above as long as the fragment retains HLA-G activity.

Preferably the second portion comprises the extracellular domain of the HLA-G molecule which comprises α1, α2, α3 and β2m. The second portion may comprise a polypeptide having the sequence given as SEQ ID NO:1 or SEQ ID NO:2. SEQ ID NO:1 is the reference HLA-G sequence with leader peptide. SEQ ID NO:2 is the HLA-G sequence used in the examples, along with the leader peptide and with a truncated transmembrane/cytoplasmic domain for synthesis of the recombinant conjugate. The second portion may comprise a polypeptide having p % sequence identity to the sequence given as SEQ ID NO:1 or 2. The percentage values of p may be 50, 55, 60, 65, 70, 75, 80, 85, 90, 91, 92, 93, 94, 95, 96, 97, 98, 99, 99.5, 99.9 or 100. References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of “Current Protocols in Molecular Biology”. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is taught in Smith & Waterman (1981).

Non-human forms of the HLA class Ib molecules, and in particular HLA-G, are known. For example, the rhesus monkey (Macaca mulatta) and olive baboon (Papio anubis) possess a novel class Ia-related locus. This gene encodes glycoproteins with characteristics that resemble those of HLA-G, including restricted tissue distribution, alternative splicing of mRNA, truncated cytoplasmic domain, and limited polymorphism. Thus, this molecule may be the functional homologue of HLA-G (Hunt and Langat, 2002 Biol Reprod. 67(5):1367-74). The second portion may comprise a non-human homologue of HLA-G.

Other molecules that may have “HLA-G activity”, i.e. bind to inhibitory receptors on immune cells and modulate their function, will be clear to those of skill in the art. This includes aptamers and even small molecules identified by screening combinatorial libraries.

The second portion may comprise a member of the HLA class Ib antigens other than HLA-G, which exhibit HLA-G activity e.g. HLA-E or HLA-F. Conventional class I HLA molecules can also bind to some of the HLA-G receptors, although the stoichiometry and the functional consequences of such binding and how it compares to that of HLA-G are not entirely clear.

Another class of molecules that may be generated to have “HLA-G activity” are antibodies specific to the receptors of HLA-G which mimic the action of the natural ligand, i.e. agonistic antibodies to ILT2, ILT4, KIR2DL4 etc. Naturally, any molecule that can bind to these receptors in a manner conducive to transmitting a signal inside the cell is endowed with “HLA-G activity”.

Linker and Other Components

The first and the second portion of the conjugate are connected. This connection may be direct or it may be via a linker. In both cases the connection serves to keep the first and second portion attached to each other.

Standard protein cross-linkers could be used to generate the conjugate of this invention. These will be familiar to those skilled in the art and involve methods which exploit the chemistry of the protein side-chains to cross link two or more polypeptides. Examples are disulphide, amine-carboxyl and amine-sulfhydryl linkers. The following website provides a useful overview: http://www.piercenet.com/products/browse.cfm?fldID=020306

The linker may be a peptide. Peptide linker sequences will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise poly glycine linkers (i.e. comprising Glyₙ where n=2, 3, 4, 5, 6, 7, 8, 9, 10 or more), and histidine tags (i.e. Hisn where n=3, 4, 5, 6, 7, 8, 9, 10 or more). Other suitable linker amino acid sequences will be apparent to those skilled in the art. A useful linker is GSGGGG or GSGSGGGG. The (Gly)4 tetrapeptide represents a typical poly glycine linker. Preferably, the linker is a glycine-serine peptide having the formula (G₄S)n, where n is any number from 1 to 100, i.e. 1, 2, 3, 4, 5, 6, 7, 8, 9, 10, 11, 12, 13, 14, 15, 16, 17, 18, 19, 20, 25, 30, 35, 40, 45, 50, 60, 70, 80, 90 or 100.

The linker may be a branched peptide linker as described for example in U.S. Pat. No. 6,759,509).

The linker may be a chemical cross-linking moiety, for example.

Conjugate

As described above, the conjugates of the invention comprise a first portion connected to a second portion. The first and second portion may be produced individually and subsequently connected to form the conjugate, or a single conjugate molecule may be produced which comprises both the first and second portion.

A preferred embodiment of a conjugate of the invention is a polypeptide which comprises the variable domains of the heavy and light chain of an anti-HLA antibody connected by a glycine-serine linker having the formula (G₄S)n to the extracellular domain of HLA-G. Examples of various forms of this preferred molecule is provided as SEQ ID NOs: 3 and 4. Fragments, functional equivalents (e.g. mutants with one or more deletions, substitutions, insertions or additions) and degenerate forms of these molecules are also included as aspects of the invention, for example, polypeptides having p % sequence identity to the sequence given as SEQ ID NO:3 or 4. The percentage values of p may be 50, 55, 60, 65, 70, 75, 80, 85, 90, 91, 92, 93, 94, 95, 96, 97, 98, 99, 99.5, 99.9 or 100.

In a preferred embodiment of the invention, the conjugate will contain a furin cleavage. The furin cleavage site could, for example, be located between two variable antibody fragments in the first portion and can resolve difficulties with protein expression. The fundamental problem addressed is that of stoichiometry: if more than one polypeptide is required to form a heterodimer together, the two polypeptide species should be present in the endoplasmic reticulum at equivalent concentrations. So if the two polypeptides are expressed on two different genes in the same or different vectors, the expression of the two genes and consequently the rate of translation and protein production may not be equivalent. This results in an excess of one of the two component polypeptides which would interfere with protein production. On the other hand, if both polypeptides are encoded on the same gene in the same vector, the linker peptide between the two polypeptides of interest may interfere with folding and function. To solve this problem, protein engineers have come up with the solution of encoding an enzymatic site between the two polypeptides of interest, e.g. furin, to cleave off the two polypeptides after translation. This provides the benefit of separating the two peptides to ensure adequate folding and function, but it ensures that they are both present in equimolar quantities for optimum production of the whole protein. Accordingly, a conjugate of the invention may be designed to incorporate technology of this kind.

A further preferred form of a conjugate according to the invention is one that is linked either covalently or non-covalently. For example, any of the first portions possessing an affinity for an MHC molecule specific to the graft that are described in detail above can be linked covalently or non-covalently to any second portion described above that possesses HLA-G activity. More specifically, an antibody or antibody fragment directed against an MHC molecule specific to the graft can be linked covalently or non-covalently to a second portion that possesses HLA-G activity, such as the extracellular portion. As described in detail above, such an antibody fragment may comprise the variable domains of the heavy and light chain of an anti-HLA antibody. Preferred forms may be linked to the extracellular domain of HLA-G, either covalently or non-covalently. An example of a non-covalent interaction would be through the use of binding partners such as biotin and streptavidin; other examples will be known to those of skill in the art.

For example, one preferred form of a conjugate of the invention is one which includes a BB7.2 or B11 monoclonal antibody linked non-covalently to HLA-G. The conjugate may be formed by mixing streptavidin-linked antibody or antibody fragment with biotinylated HLA-G. Different ratios of antibody and HLA-G moiety may be used, for example, between 1:20 and 1:2; molar ratios of both 1:12 or 1:4 have been tested herein and both found to be effective.

Polypeptides

The first portion and/or the second portion of the conjugate may be a polypeptide. In addition the entire conjugate may be a polypeptide.

Polypeptides can be prepared in various forms (e.g. native, fusions, glycosylated, non glycosylated, myristoylated, non myristoylated, lipdated, non lipidated, monomeric, multimeric, particulate, denatured, etc.).

Polypeptides may comprise a detectable label (e.g. a radioactive or fluorescent label, or a biotin label).

Polypeptides can be prepared in many ways e.g. by chemical synthesis (at least in part), by digesting longer polypeptides using proteases, by translation from RNA, by purification from cell culture (e.g. from recombinant expression), from the organism itself (e.g. isolation from tissue), from a cell line source etc.

The term “polypeptide” refers to amino acid polymers of any length. The polymer may be linear or branched, it may comprise modified amino acids, and it may be interrupted by non amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogues of an amino acid (including, for example, unnatural amino acids, etc.), as well as other modifications known in the art. Polypeptides can occur as single chains or associated chains.

Polypeptides can be naturally or non-naturally glycosylated (i.e. the polypeptide has a glycosylation pattern that differs from the glycosylation pattern found in the corresponding naturally occurring polypeptide).

The polypeptides may also comprise additional groups to assist in the purification, solubilisation, chromatography, imaging, enzyme or chemical modification of the polypeptide.

The polypeptides may also form part of a larger polypeptide. For example, a polypeptide may be flanked by additional n-terminal and/or c-terminal amino acids, for example, a his-tag or fused to GST.

In general, the polypeptides are provided in a non-naturally occurring environment, i.e. they are separated from their naturally occurring environment.

Nucleic Acids

Where the conjugate is a polypeptide, the invention provides a nucleic acid encoding the conjugate of the invention.

The invention also provides a nucleic acid comprising a nucleotide sequence with n % or more sequence identity to a nucleic acid of the invention. The percentage values of n may be 50, 55, 60, 65, 70, 75, 80, 85, 90, 91, 92, 93, 94, 95, 96, 97, 98, 99, 99.5, 99.9 or 100.

Nucleic acids of the invention are preferably provided in isolated or substantially isolated form i.e. substantially free from other nucleic acids (e.g. free from naturally-occurring nucleic acids), generally being at least about 50% pure (by weight), and usually at least about 90% pure.

Nucleic acids of the invention can take various forms.

Nucleic acids of the invention may be single-stranded or double-stranded. Unless otherwise specified or required, any embodiment of the invention that utilizes a nucleic acid may utilize both the double-stranded form and each of two complementary single-stranded forms which make up the double stranded form.

Nucleic acids of the invention may be circular or branched, but will generally be linear.

Nucleic acid of the invention may be attached to a solid support (e.g. a bead, plate, filter, film, slide, microarray support, resin, etc.)

For certain embodiments of the invention, nucleic acids are preferably at least 100 nucleotides in length (e.g. 100, 110, 120, 130, 140, 150, 160, 170, 180, 190, 200, 225, 250, 275, 300, 350, 400, 500, 750, 1000, 1500, 2000 nucleotides or longer).

For certain embodiments of the invention, nucleic acids are preferably at most 2000 nucleotides in length (e.g. 1900, 1500, 1000, 500, 450, 400, 350, 300, 250, 200, 150, 140, 130, 120, 110, 100 nucleotides or shorter).

Nucleic acids of the invention may carry a detectable label e.g. a radioactive or fluorescent label, or a biotin label.

Nucleic acids of the invention can be prepared in many ways e.g. by chemical synthesis (at least in part), by digesting longer nucleic acids using nucleases (e.g. restriction enzymes), by joining shorter nucleic acids (e.g. using ligases or polymerases), from genomic or cDNA libraries, etc.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. The term “nucleic acid” is not intended to be limiting as to the length or structure of a nucleic acid unless specifically indicated, and the following are non-limiting examples of nucleic acids: a gene or gene fragment, mRNA, tRNA, rRNA, ribozymes, cDNA, recombinant nucleic acids, branched nucleic acids, plasmids, vectors, DNA from any source, RNA from any source, probes, and primers. Where nucleic acid of the invention takes the form of RNA, it may have a 5′ cap.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T). The terms also imply a direction—the complement of 5′ ACAGT-3′ is 5′ ACTGT-3′ rather than 5′ TGTCA-3′.

Nucleic acids of the invention can be used, for example: to produce polypeptides; or to generate additional copies of the nucleic acids.

References to a percentage sequence identity between two nucleic acid sequences mean that, when aligned, that percentage of bases are the same in comparing the two sequences. This alignment and the percent homology or sequence identity can be determined using software programs known in the art, for example those described in section 7.7.18 of “Current Protocols in Molecular Biology”. A preferred alignment program is GCG Gap (Genetics Computer Group, Wisconsin, Suite Version 10.1), preferably using default parameters, which are as follows: open gap=3; extend gap=1.

Where a nucleic acid is said to “encode” a polypeptide, it is not necessarily implied that the polynucleotide is translated, but it will include a series of codons which encode the amino acids of the polypeptide.

Vectors

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector.

The vector of the invention is preferably an autonomously replicating episomal or extrachromosomal vector, such as a plasmid.

The vector of the invention preferably comprises an origin of replication. It is preferred that the origin of replication is active in prokaryotes but not in eukaryotes.

The vector of the invention may comprise a multiple cloning site.

Host Cells

A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

In a preferred embodiment of the invention, the first and second portions of the conjugate, along with any linker regions, cleavage sites or tags which may be included, are expressed as a direct in-frame translated protein.

In another embodiment of the invention the first and second portions of the invention may be generated as separate entities then bonded together using biological or chemical techniques.

Any cleavage sites or tags which may be included could be expressed as an in frame translational fusion with either the first or second portions, or may be bonded to the conjugate using biological or chemical techniques.

Pharmaceutical Compositions.

The invention provides a pharmaceutical composition comprising conjugates or nucleic acids of the invention.

Pharmaceutical compositions encompassed by the present invention include as active agent, the conjugates or nucleic acids of the invention disclosed herein in a therapeutically effective amount. An “effective amount” is an amount sufficient to effect beneficial or desired results, including clinical results. An effective amount can be administered in one or more administrations. For purposes of this invention, an effective amount is an amount that is sufficient to palliate, ameliorate, stabilize, reverse, slow or delay the symptoms and/or progression of graft rejection. Additionally, an effective amount is an amount that is sufficient to induce tolerance to a graft.

The compositions can be used to treat and/or prevent graft rejection as well as to induce tolerance to a graft. In addition, the pharmaceutical compositions can be used in conjunction with conventional methods of immunosuppresion.

The terms “treatment”, “treating”, “treat” and the like are used herein to generally refer to obtaining a desired pharmacologic and/or physiologic effect. The effect may be prophylactic in terms of completely or partially preventing graft rejection or a symptom thereof and/or may be therapeutic in terms of a partial or complete stabilization or cure for graft rejection and/or adverse effect attributable to graft rejection.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg of the compositions of the present invention in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol.

Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier.

Pharmaceutically acceptable salts can also be present in the pharmaceutical composition, e.g. mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in reference.

The composition is preferably sterile and/or pyrogen-free. It will typically be buffered at about pH 7.

Once formulated, the compositions contemplated by the invention can be (1) administered directly to the subject; or (2) delivered ex vivo, to the graft. For example, prior to the graft being surgically implanted in the recipient it may be treated with a pharmaceutical composition of the invention. This may be achieved by washing or bathing the graft in a solution containing a pharmaceutical composition of the invention. Such a washing, or bathing step may involve the graft being immersed in, or coated with, a pharmaceutical composition of the invention for a specified period of time, under appropriate conditions. Examples of suitable bathing times include 1, 2, 3, 4, 5, 6 or more hours, preferably at around 4° C. Conjugate according to the invention could be incorporated into known solutions of the type used to collect and preserve haematopoietic stem cells before transplantation.

The graft may be warmed up to body temperature before use.

In one example, a pharmaceutical composition according to the invention can be incorporated into a topical preparation. Such a possibility would be well suite for “painting” the skin in graft-versus-host disease (GVHD), as skin is often one of the affected target organs. A formulation specific for burn sites could be used to alleviate inflammation and prepare the burn site for an allogeneic skin graft.

Similarly, a formulation of the composition may be designed to administer at high concentration in a given location via intravascular catheters, used, for example, for targeting the liver or intestine in GVHD.

Direct delivery of the compositions will generally be accomplished by parenteral injection, e.g. subcutaneously, intraperitoneally, intravenously or intramuscularly, intralesional intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

The dose and the means of administration of the inventive pharmaceutical compositions are determined based on the specific qualities of the therapeutic composition, the condition, age, and weight of the patient, the extent of graft rejection, and other relevant factors.

Gene therapy with a nucleic acid encoding a suitable form of a conjugate according to the invention is another possibility for safe delivery. Gene therapy can either be applied to the graft itself before transplantation (for solid vascularised grafts), to cells in suspension in case of stem cell transplantation or to the donor before or after transplantation. Modifications of the vector used for gene therapy could be used to control expression of the gene by exogenously administered drugs. The application of gene therapy could either be by injection of the vector DNA intramuscularly or by using devices such as gene guns (biolistics).

Methods of Treatment

The invention includes a method of preventing graft rejection in a patient comprising administering a conjugate or a pharmaceutical composition of the invention to the patient. As described in detail above, in order to be suitable as an acceptor patient, the patient must have an MHC Class I allele that is different to that recognised by the conjugate of the invention. Accordingly, the availability of a conjugate with specificity for MHC Class I poses the only restriction on the extent to which this invention may be utilised across multiple populations. It is worth noting thought that restrictions are posed only on the potential donor pool, but not on the recipients, i.e. anyone can be a recipient, but only those who possess a tissue type recognised by a conjugate according to the invention can become donors. This poses no practical difficulty as conjugates according to the invention can cover all common tissue types. Accordingly, however, a method according to the invention may incorporate a step of testing the MHC allele (such as the MHC Class I allele) of one or both of the donor and recipient.

The invention also includes a method of inducing tolerance to a graft in a patient comprising administering a conjugate or a pharmaceutical composition of the invention to the patient.

The conjugate or pharmaceutical composition may be administered to the patient before they undergo transplant surgery, during the transplant surgery and/or after the transplant surgery has been completed.

The invention also provides the use of conjugates and/or pharmaceutical in the methods of treatment described above.

A patient to be treated with the methods of the invention will have undergone graft transplantation or will be being prepared for transplant surgery. The patient will be an animal, preferably a mammal and more preferably a human.

Common grafts are liver, kidney, pancreas, cornea, heart, bone marrow, skin and stem cells. Stem cell transplantation is gaining credibility as a viable method of restoring cellular and tissue function in cases of disease or deficiency. Other examples will be known to those of skill in the art.

Tolerance, once induced, may be sustained for as long as the graft continues to be present in the recipient as this would ensure sustained supply of the alloantigen, so maximising the chances of maintaining immunological tolerance. The situation thus provides a positive feedback loop whereby tolerance leads to graft integrity preservation which leads to more tolerance by continued supply of the alloantigen. In the long term, withdrawal of a conjugate according to the invention could be envisaged when the immunological tolerance becomes self-sustaining. Naturally, this cycle could be broken if due to non-immunological causes, the graft is damaged which could result in inflammation and may threaten to re-ignite the alloreactive processes. In this case, re-commencing therapy may be necessary to protect the graft.

The invention will now be described in detail, with particular reference to examples. It will be appreciated that these examples are in no way intended to be limiting.

### BRIEF DESCRIPTION OF FIGURES

FIG. 1—Schematic representation of direct allo-sensitisation of CD4 and CD8 T-cells.

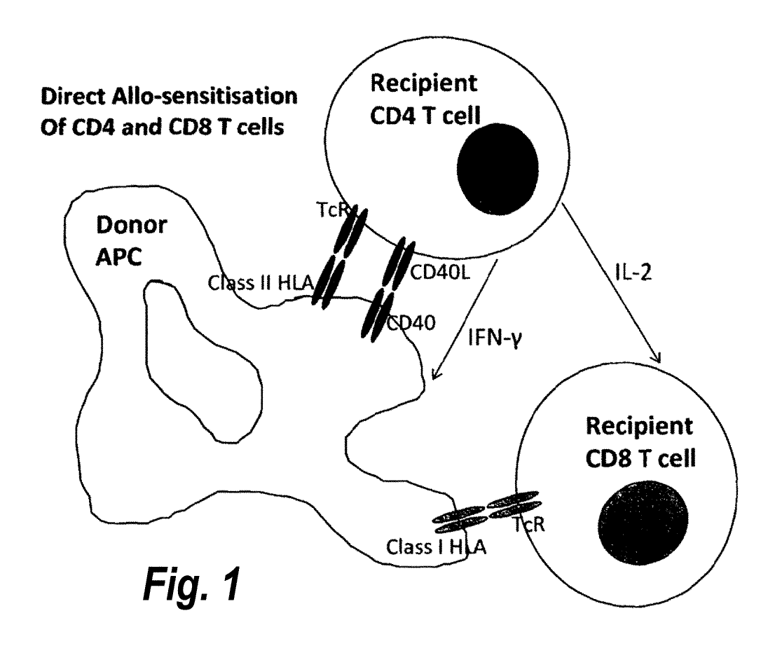

FIG. 2—Schematic representation of CD4-independent CD8 direct allo-sensitisation.

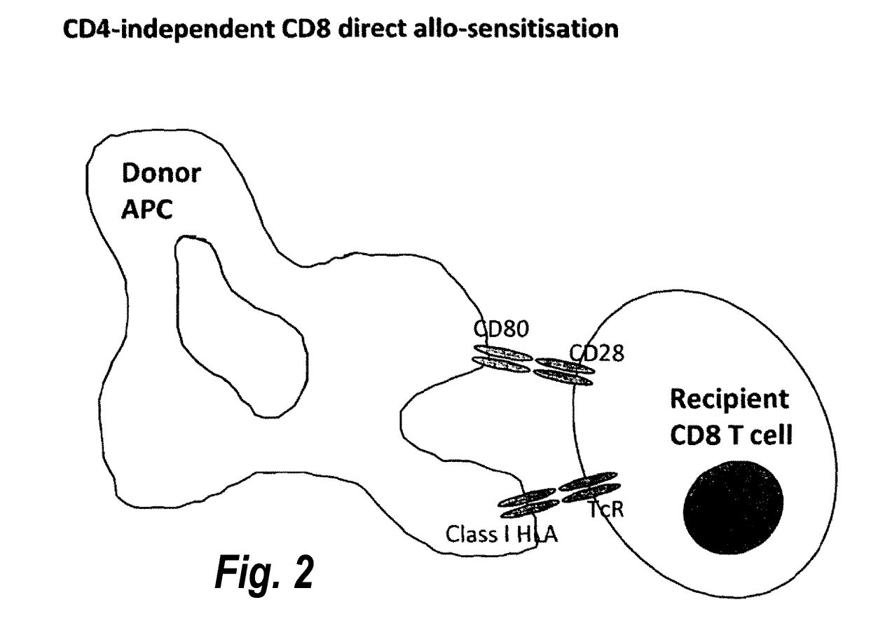

FIG. 3—Schematic representation of indirect allo-sensitisation of CD4 and CD8 cells.

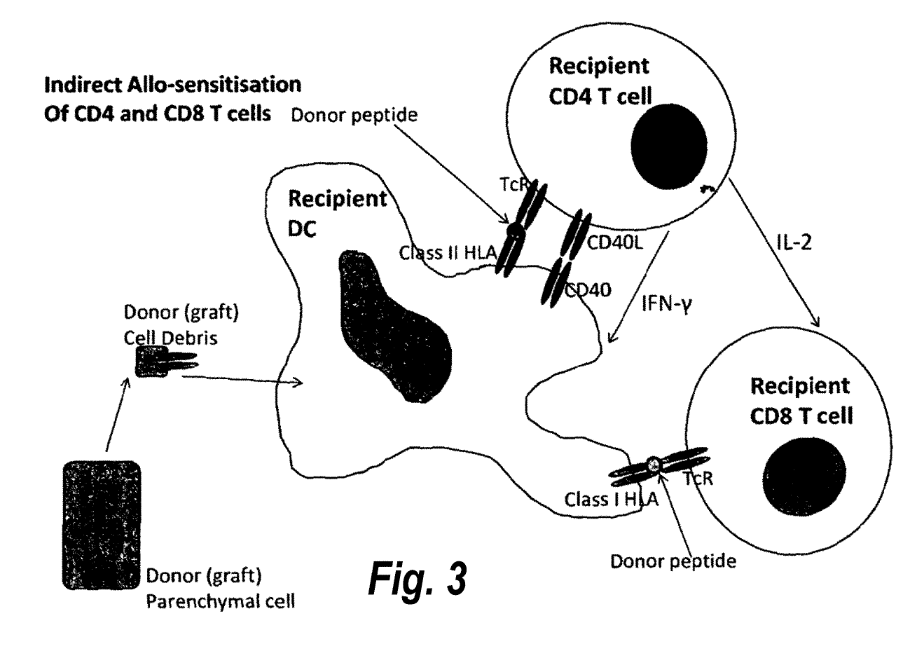

FIG. 4—Schematic representation of inhibition of direct allo-sensitisation of CD4 and CD8 T cells by the conjugate.

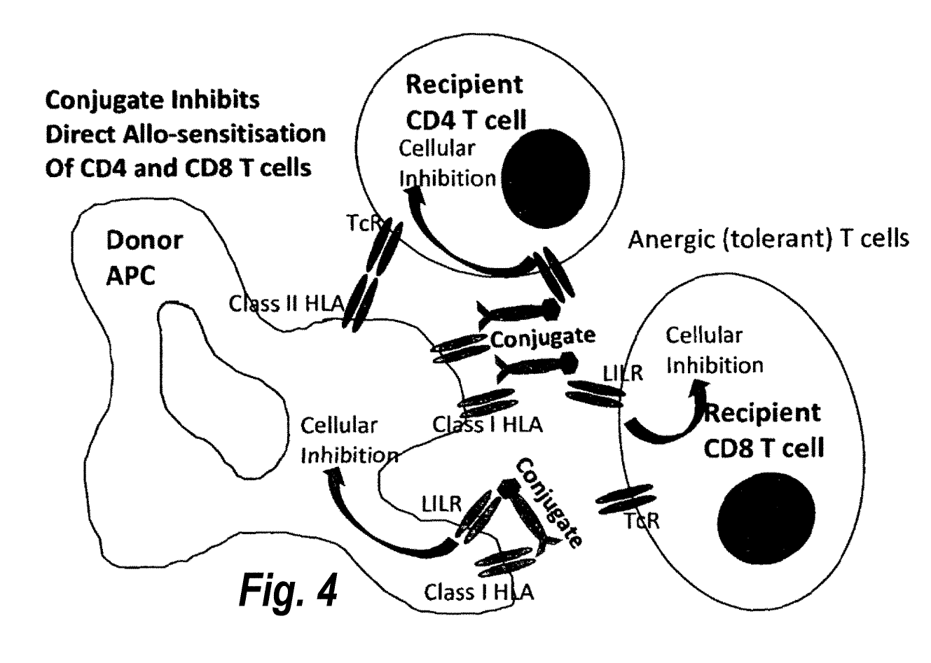

FIG. 5—Schematic representation of inhibition of CD4-independent CD8 direct allo-sensitisation by the conjugate.

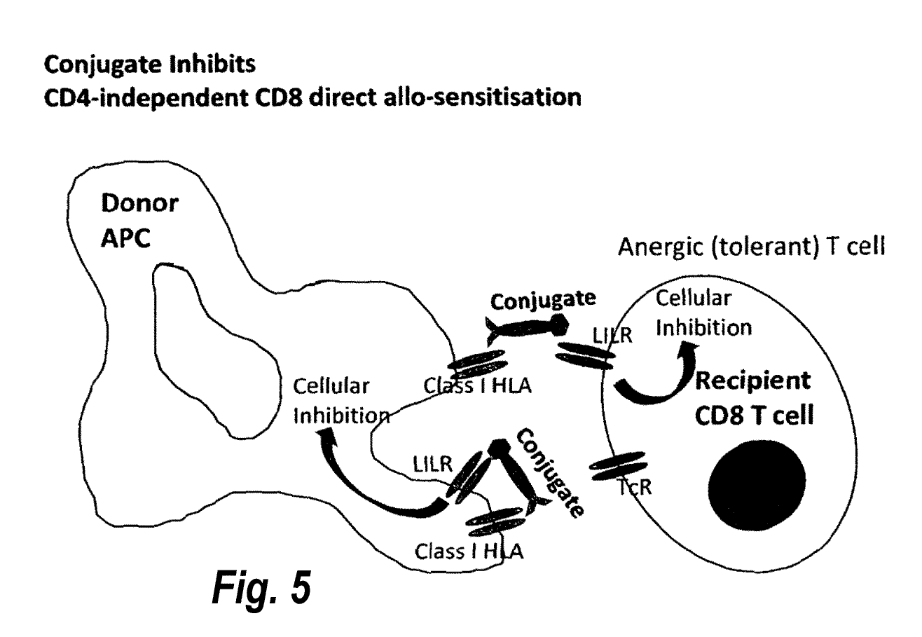

FIG. 6—Schematic representation of inhibition of induction of regulatory T cells with direct allo-recognition by the conjugate.

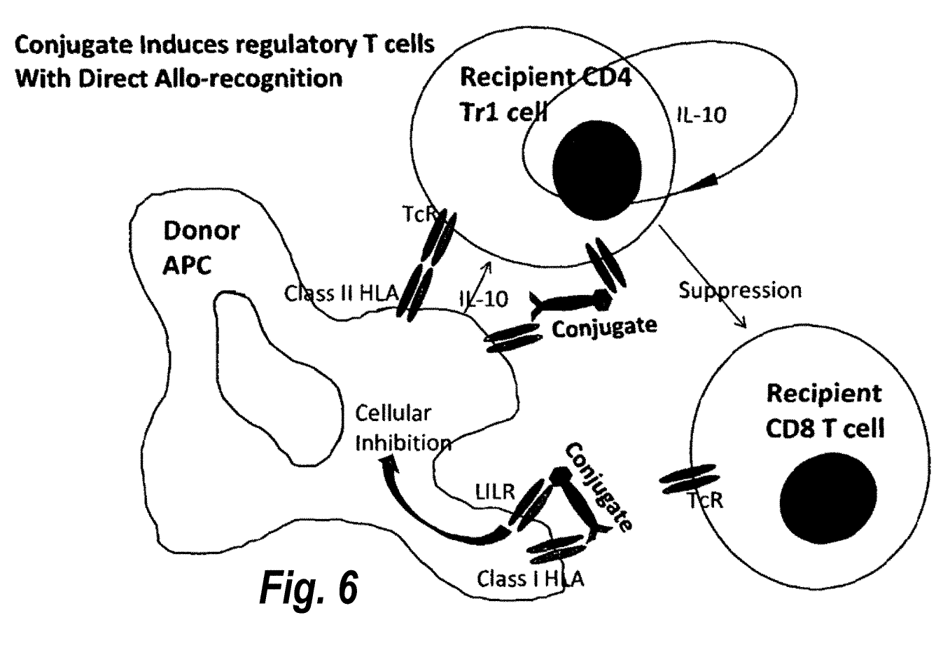

FIG. 7—Schematic representation of inhibition of inhibition of indirect allo-sensitisation of CD4 and CD8 T cells by the conjugate.

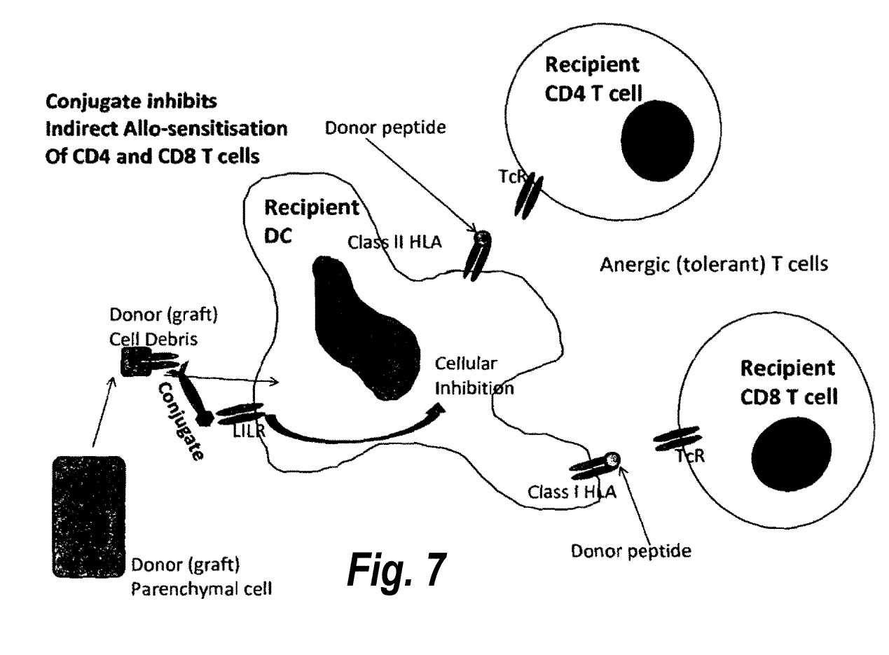

FIG. 8—Schematic representation of inhibition of induction of regulatory T cells with indirect allorecognition by the conjugate.

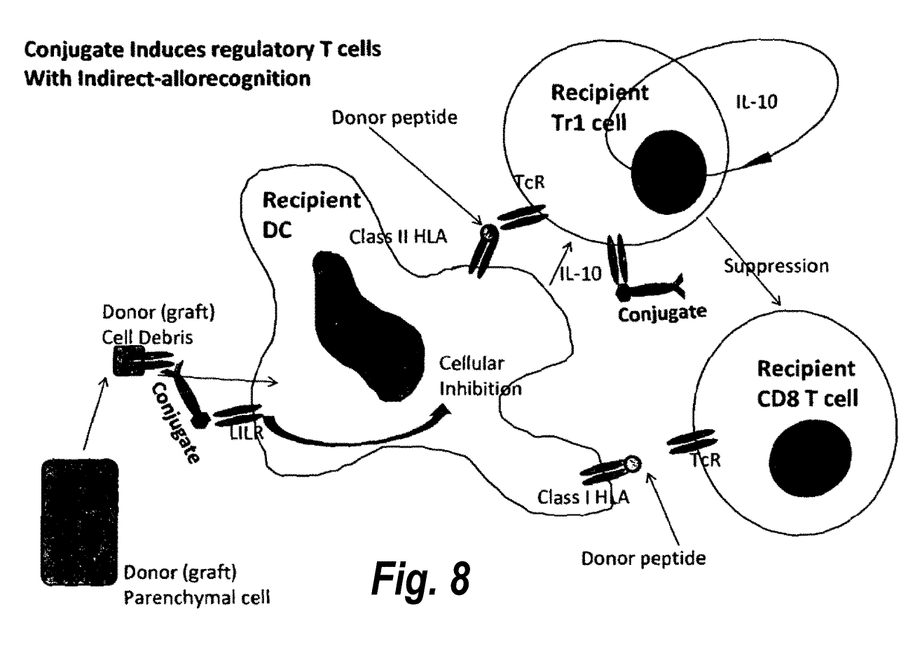

FIG. 9—Schematic representation of conjugate A. α1, α2, α3 and β2m are the extracellular domains of the HLA-G molecule. VLA and VHA are the variable domains of the heavy and light chains of the monoclonal antibody.

FIG. 10—Schematic representation of conjugate B; α1, α2, α3 and β2m are the extracellular domains of the HLA-G molecule. VLA and VHA are the variable domains of the heavy and light chains of the monoclonal antibody.

FIG. 11—Transient transfection control.

FIG. 12—Western blot showing conjugate B in COS7 cell supernatant. Lane 1=vector only transfectant; lane 2=HLA-G/β2m transfectant; lane 3=conjugate B transfectant.

FIG. 13—Mixed lymphocyte reaction in the presence and absence of conjugate C. Responder cells are HLA-A2 negative, stimulator cells are HLA-A2 positive.

FIG. 14—Western blot from immunoprecipitation demonstrating conjugate B binding to HLA-A2. Lane 1=SA beads incubated with conjugate B supplemented; lane 2=SA beads+bHLA-A2 incubated with conjugate B; lane=SA beads+bHLA-A2 alone. Detection antibody for Western blot 4H84 (anti-HLA-G).

FIG. 15—Western blot from immunoprecipitaion demonstrating binding of conjugate B, not conjugate A to HLA-A2. Lane 1=SA beads/BB7.2; lane 2=SA beads/bHLA-A2/BB7.2; lane 3=SA beads/conjugate B supplemented; lane 4=SA beads/bHLA-A2/conjugate B supplemented; lane 5=SA beads/bHLA-A2; lane 6=SA beads/bHLA-A2/conjugate B supplemented; lane 7=HLA-G/β2m supplemented; lane 8=Conjugate B supplemented. Detection antibody for Western blot 4H84 (anti-HLA-G).

FIGS. 16A-16B—Western blot from immunoprecipitation showing binding of anti HLA-G monoclonal antibodies (depicted in FIG. 16A) to conjugate B and HLA-G/β2 microglobulin (depicted in FIG. 16B).

FIG. 16A lane 1=protein G alone; lane 2=protein G+MEM-G9 mAb; lane 3=Protein G+MEM-G9 incubated with conjugate B supplemented; lane 4=Protein G+87G-PE mAb; lane 5=Protein G+87G-PE incubated with conjugate B supplemented; lane 6=Protein G+4H84 mAb; lane 7=Protein G+4H84 incubated conjugate B supplemented. Detection antibody: 4H84 (anti HLA-G). Circled band in lane 7 shows 4H84 binding to conjugate B.

FIG. 16B lane 1=Protein G+MEM-G9; lane 2=Protein G+MEM-G9 incubated with HLA-G+β2m supplemented; lane 3=Protein G+87G-PE; lane 4=Protein G+87G-PE incubated with HLA-G+β2m supplemented; lane 5=Protein G+4H84; lane 6=Protein G+4H84 incubated with HLA-G+β2m supplemented; lane 7=Protein G/W6/32; lane 8=Protein G+W6/32 incubated with HLA-G+β2m supplemented. Detection antibody: 4H84. Circled bands in lanes 2, 4 and 8 show binding. Lane 6 also appears to show binding however this is slightly obscured by non-specific band of similar molecular weight.

FIG. 17—FACS analysis of conjugate C binding to HLA-A2 positive lymphocytes.

FIG. 18—FACS analysis of conjugate C binding to HLA-A2 negative monocytes (binding to ILT4).

FIG. 19—FACS analysis showing no binding of conjugate C to HLA-A2 negative lymphocytes.

FIG. 20—Schematic for the ORIGBv2 and B11GBv2 constructs which consist of the HLA-G heavy chain linked to the anti-HLA-A2 ScFv fragment; with the β2m being co-transfected on a separate plasmid.

FIG. 21—Western blot showing immunoprecipitation using biotinylated HLA-A2 bound to streptavidin beads. The primary detection antibody is 4H84 (non-conformational anti-HLA-G). Faint binding of ORIGBv2 (predicted molecular weight of 62 kDa) and stronger binding of B11GBv1 (predicted molecular weight of 73 kDa) and B11GBv2 (predicted molecular weight of 60 kDa) is seen.

FIG. 22—Western blot showing immunoprecipitation using anti-HLA-G (87G) bound to protein G beads. The primary detection antibody is 4H84 (non-conformational anti-HLA-G). Faint binding of B11GBv1 (predicted molecular weight of 73 kDa) and stronger binding of ORIGBv2 and B11GBv2/β2m (predicted molecular weight of 62 and 60 kDa respectively) is seen in the region of the box.

FIGS. 23A1-23D—G-Body dose-dependently inhibits CD3/CD28-mediated proliferation of HLA-A2 positive but not that of HLA-A2 negative PBMC. PBMC (1×10⁵) were activated with beads coated with anti-CD3/anti-CD28 at a ratio of 1 bead to 5 cells in the absence or the additional presence of increasing doses of G-Body. Proliferation was assessed by the incorporation of [³H]-TdR. (FIG. 23A1 and FIG. 23A2) HLA-A2 positive donor C, proliferation measured at day 3 (FIG. 23A1) or day 7 (FIG. 23A2). (FIG. 23B) HLA-A2 positive donor J, proliferation measured at day 3. (FIG. 23C) HLA-A2 negative donor K, proliferation measured at day 7. $ Controls used: anti-HLA-A2 and HLA-G-tetramer, tested at the highest concentration, equivalent to that present in 5 ug/ml G-Body. (FIG. 23D) HLA-A2 negative donor I, proliferation measured at day 7. ^{$} Controls used: anti-HLA-A2 and HLA-G-tetramer, tested at the highest concentration, equivalent to that present in 5 ug/ml G-Body.

FIGS. 24A1-24C—G-Body dose-dependently inhibits superantigen (SEB)-mediated proliferation of HLA-A2 positive but not that of HLA-A2 negative PBMC. PBMC (1×10⁵) were activated with 1 ng/ml SEB unless indicated in the absence or the additional presence of increasing doses of G-Body. Proliferation was assessed by the incorporation of [³H]-TdR. (FIG. 24A1 and FIG. 24A2) HLA-A2 positive donor J, proliferation measured at day 3 (FIG. 24A1) or day 7 (FIG. 24A2). (FIG. 24B) HLA-A2 positive donor C, proliferation measured at day 3. (FIG. 24C) HLA-A2 negative donor K, proliferation measured at day 3. ^{$} Controls used: isotype control for anti-HLA-A2, anti-HLA-A2 and HLA-G-tetramer, tested at the highest concentration, equivalent to that present in 5 ug/ml G-Body.

### EXAMPLES

### Example 1—Generation of Peptides

Sequencing of BB7.2 Hybridoma

Total RNA was extracted from BB7.2 hybridoma cell pellets using the Fusion Antibodies Ltd. in house total RNA extraction protocol. The extracted mRNA was reverse transcribed to generate cDNA using an oligo (dT) primer. Ths cDNA was used as a template for PCR reactions using oligonucleotide primers flanking the VH and VL regions of the monoclonal antibody.

The PCR-amplified VH and VL DNA products were cloned into the pCR2.1 sequencing vector (Invitrogen) and transformed into TOP10 E. coli cells. Positively transformed colonies were isolated and sequenced by Fusion Antibodies Ltd.

Conjugate a Comprising the BB7.2 Hybridoma ScFv

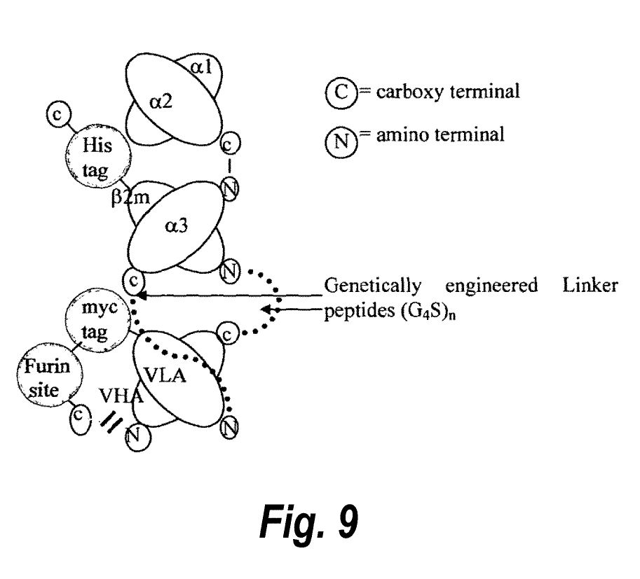

The polypeptide conjugate (denoted conjugate A) comprising HLA-G, β2-microglobulin and the ScFv portion of BB7.2 was generated as two polypeptide chains. The first comprised the HLA-G fused to the VH domain of BB7.2 and the second comprised the VL domain of BB7.2 fused to β2-microglobulin. Each fragment of each polypeptide chain was linked with a (G₄S)₁₋₃ linker. The two polypeptides were linked with a furin cleavage site linker and c-myc and 6×His tags were incorporated to allow for purification and detection. Various restriction enzyme cleavage sites were incorporated in the synthetic genes to enable removal and/or replacement of various components and to allow future modification (FIG. 9).

Following their synthesis, the construct was cloned into a pcDNA3.1 expression vector by Genscript. Competent E. coli cells were then transformed with the pcDNA3.1 vector containing the construct and positive transformants were identified by the acquisition of antibiotic resistance. Plasmid DNA was obtained by maxipreparation.

Conjugate B Comprising the B11 ScFv

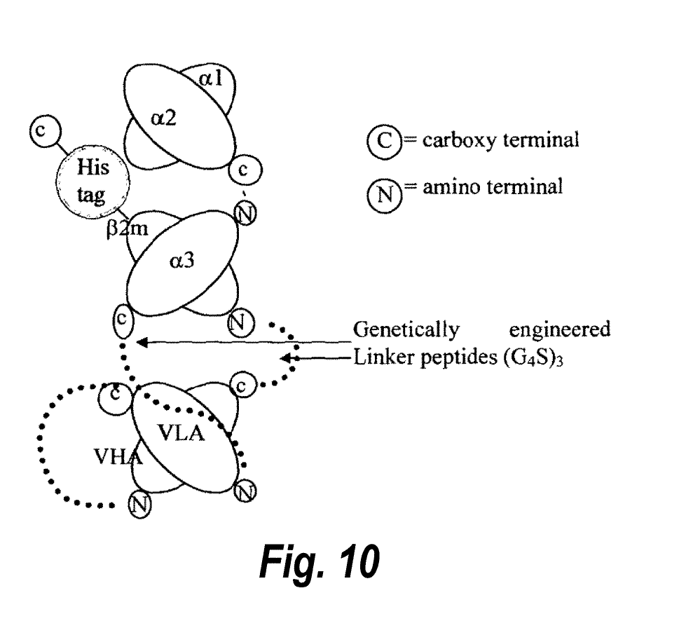

A second conjugate (denoted conjugate B) was generated from conjugate A by substituting the ScFv sequence derived from BB7.2 with the published B11 sequence (FIG. 10).

A B11 ScFv clone was obtained as a kind gift from Dr. Watkins and Dr. Ouwehand, Cambridge University, UK. After verification of the published sequence, the clone was transformed into TOP10 cells followed by plasmid DNA extraction. Plasmid DNA was sequenced by Geneservice using M13F, M13R and custom “AGGCGAGTCAGGACATTAGC” primers. Sequence verification was performed by comparison with the published sequence.

Design and Synthesis of the Custom B11 ScFv Inserted Sequence

The custom B11 ScFv sequence comprised the verified B11 ScFv sequence with (G₄S)₃ linkers attached at the 5′ and 3′ ends. (G₄S)₃ linkers were also incorporated between the two variable domains. BspEI and BamHI restriction enzyme cleavage sites were incorporated at the 5′ and 3′ ends to allow cloning of the custom B11 ScFv sequence into the conjugate A construct in place of the BB7.2 ScFv sequence. Conjugate B did not include the c-myc tag or the furin cleavage site.

This custom sequence was submitted to GenScript, where the codons were optimised for expression in COST cells. The custom B11 ScFv polynucleotide was synthesized and cloned into a pUC57 vector prior.

Cloning the Custom B11 ScFv Sequence into Construct A

The B11 pUC57 vector was transformed into TOP10 cells followed by plasmid minipreparation. Conjugate A and the custom B11 pUC57 plasmids were digested with BspEI and BamHI for 60 minutes at 37° C. The digest products were separated on 0.8% agarose gel. The digested conjugate A fragment (without the BB7.2 ScFv) and the custom B11 ScFv sequence were obtained by gel extraction then ligated. TOP10 cells were then transformed with the ligation product before plasmid DNA minipreparation was performed.

The plasmid DNA minipreparations were sequenced by Geneservice. Using T7F, bGHR and “ATGGAACCTTCCAGAAGTGG” primers, sequence analysis confirmed that the custom B11 ScFv sequence had correctly substituted the BB7.2 of conjugate A.

Following sequence confirmation, a maxipreparation was performed using the transformed TOP10 cells.

Cloning of Furin for Co-Transfection with Conjugate A to Enhance Furin Cleavage

A pRc/CMV plasmid with a furin cDNA insert was obtained from a collaborator. This plasmid was transformed into TOP10 cells and a maxipreparation was generated.

Conjugate C Comprising Biotin-Streptavidin Joined Polypeptides

A chemically joined conjugate (denoted conjugate C) was generated which included the BB7.2 monoclonal antibody linked to HLA-G. The conjugate was formed by mixing streptavidin-linked BB7.2 mAb with biotinylated HLA-G at a molar ratio of either 1:12 or 1:4 followed by incubation at room temperature for 30 minutes.

Conjugation of the BB7.2 Monoclonal Antibody to Streptavidin

Either 100 μg or 300 μg of purified BB7.2 mAb in PBS was added to 100 μg of streptavidin at a molar ratio of approximately 1:3 or 1:1, after addition of proprietary reagents from the EZ-Lightning Link kit. The mixture was incubated for 3 hours then the reaction was stopped using the proprietary quenching reagent.

Biotinylation of HLA-G

Biotinylated HLA-G was purchased from the Frank Hutchinson Cancer Research Center.

### Example 2—Generation of Controls

Generation of Transfection Control

An enhanced green fluorescent protein (EGFP) sequence was cloned into pcDNA3.1 then extracted by digestion using BamHI/XhoI restriction enzymes. pcDNA3.1 was also digested with BamHI/XhoI. Following separation by agarose gel electrophoresis and gel extraction, the EGFP sequence and linearised pcDNA3.1 vector were ligated together using T4 DNA ligase. TOP10 cells were then transformed with this vector. The sequence of EGFP was confirmed by sequence analysis performed at Geneservice. A plasmid maxipreparation was then performed.

Generation of Experimental Controls

Full-length HLA-G, soluble HLA-G or β2-microglobulin sequences were cloned into pcDNA3.1 for use as experimental controls

Full-length HLA-G cDNA was derived from reverse transcribed RNA extracted from JEG3 cells. Primers were designed for use with the pcDNA3.1 directional TOPO cloning system (Invitrogen) in accordance with the manufacturer's instructions. TOP10 cells were transformed with the vector containing the HLA-G full length cDNA sequence. The full length HLA-G cDNA sequence was confirmed by sequence analysis followed by maxipreparation of the vector.

Soluble HLA-G was cloned by PCR from the Conjugate A plasmid. PCR primers incorporated BamHI and XhoI restriction sites to facilitate cloning of soluble HLA-G into pcDNA3.1. To clone soluble HLA-G into pcDNA3.1, the PCR products and the pcDNA 3.1 vector were digested with BamHI/XhoI. PCR products were purified using the Qiagen Qiaquick PCR purification kit and the digested pcDNA3.1 was gel extracted following separation by agarose gel electrophoresis.

The vector and insert were ligated and used to transform TOP10 cells before DNA sequence verification and maxipreparation.

β2 microglobulin was cloned by PCR from the ‘β2M MGC’ clone obtained from ATCC. Primers were designed with BamHI and XhoI restriction enzyme sites at either end to facilitate cloning into pcDNA3.1 using the same strategy as was employed for the soluble HLA-G clone.

### Example 3—Transfection Experiments

Transient Transfection

Transient transfection of COS7 cells was performed using Superfect and subsequently Attractene reagent (purchased for use with serum-free medium).

COS7 Transfection Control

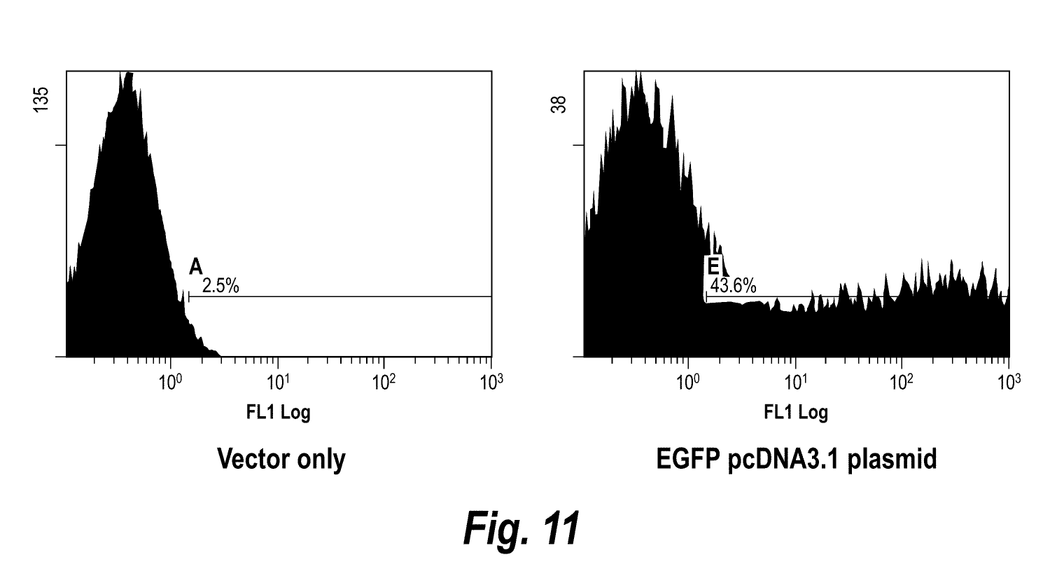

COS7 cells were transiently transfected with EGFP constructs to test whether the transfection system was working. 48 to 72 hours after transfection, COS7 cells were harvested by trypsinisation. Cells were then analysed by flow cytometry which revealed that 40-50% of COS7 cells were expressing EGFP (FIG. 11).

COS7 cells were also transfected with conjugate A or HLA-G/β2-microglobulin or were also co-transfected with conjugate A with furin to enhance cleavage of the furin cleavage site. Supernatants from these three experiments were collected at 72 hours after transfection for use in downstream applications.

Stable Transfection of K562 Cells with, ILT2 and ILT4 Plasmids

ILT2 and ILT4 plasmids were mixed with Attractene transfection reagent and incubated with K562 cells for 48 hours before being transferred to complete medium containing G418 800 μg/ml to enable the selection of selection of transfected K562 cells. Cells were maintained in selective medium for 2 to 3 weeks to ensure the absence of un-transformed cells.

Determination of Expression Levels in Transformed K562 Cells

ITL2 and ITL4-tranformed K562 cells were then stained with anti-ILT2 and anti-ILT4 mAbs respectively to determine expression levels. Highly expressing clones were subsequently sorted using a BD FACS ARIA cell-sorter prior to limiting dilution cloning.

Highly expressing clones were cultured for several days to increase their numbers before undertaking limiting dilution cloning. Cells were counted and the concentration adjusted to 2.5 cells per ml. 200 μl of cells were then incubated in 96 well flat bottom plates for 2 to 3 weeks until colonies formed. These single clone colonies were then further expanded and screened for expression of ILT2 and ILT4 to further enrich for the highest expressing cells.

Western Blotting to Confirm Expression of Proteins in COS7 Cells

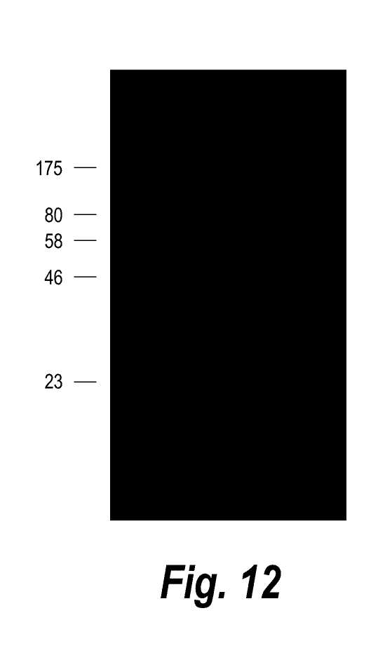

SDS-PAGE followed by Western blotting with anti-HLA-G mAb (4H84) was performed to detect presence of conjugate A and conjugate B and HLA-G in the supernatant of transfected COS7 cells. This analysis revealed the presence of protein of appropriate molecular weight (FIG. 12).

Western blotting with anti-C-myc, anti-His and anti-β2 microglobuolin antibodies failed to show clear binding, possible due to refolding of protein on membrane.

### Example 4—Mixed Lymphocyte Reactions

Cell-Mediated Toxicity

To assay cell-mediated cytotoxicity PBMCs and C073 were incubated in a ratio of 10:1 for 7 days. After 7 days, the PBMCs were washed and counted. Fresh C073 cells were painted with carboxyfluorescein succinimidyl ester (CFSE) and the sensitized PBMCs were added to the painted C073 cells in various ratios. Flow cytometric analysis showed about 70 to 80% lysis of the target cells.

Assessment of Proliferative Responses in the Presence of Conjugate C

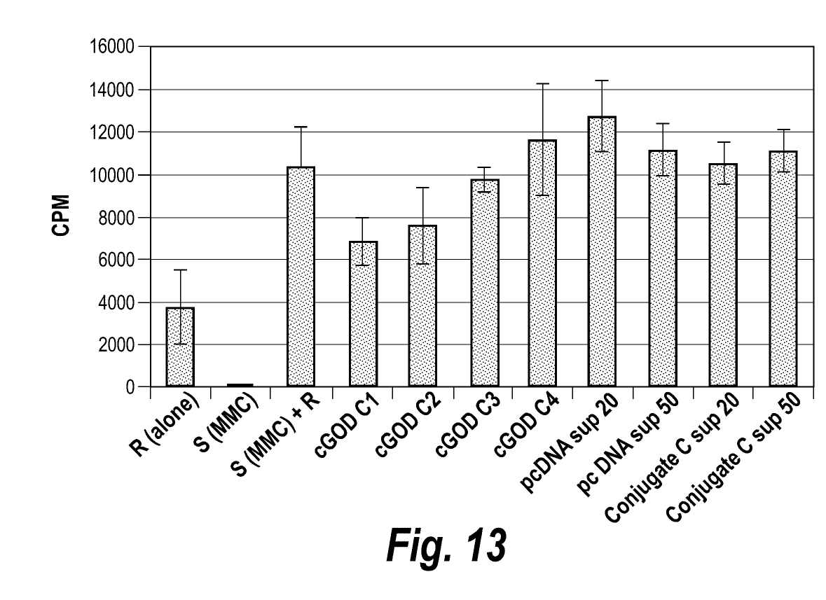

Mixed lymphocyte reactions (MLRs) with HLA-A2 negative responder and HLA-A2 positive stimulator cells treated with mitomycin-C were used to assess the effect of conjugate C on proliferative responses (FIG. 13). Responder and stimulator populations were incubated at a 1:1 ratio with 10⁵ cells per well in a 96-well U-bottomed plate, also containing RPMI medium, 10% heat inactivated foetal (bovine) calf serum (FCS), penicillin and streptomycin.

Mitomycin-C treatment involved resuspending the cells in complete medium at a concentration of 2×10⁶ cells/ml and the addition of mitomycin-C at a concentration of 25 ug/ml. Cells were incubated for 1 hour at 37° C. and then washed 3 times with complete medium prior to use.

Conjugate C was added at 4 different concentrations (C1=2.2 μg/ml, C2=1.1 μg/ml, C3=0.55 μg/ml, C4=0.22 μg/ml) with a 10-fold difference between the highest and lowest concentration. Cells were incubated for 7 days and pulsed with ³H thymidine 18 hours prior to harvesting at which point radioactivity was measured using a beta counter.

Initial experiments showed that addition of conjugate C resulted in enhanced proliferation compared to controls. Consequently, enodotoxin levels were measured using the Lonza LAL kit (because the source of the biotinylated HLA-G was bacterial). Endotoxin levels were found to exceed the upper limit of detection of the kit.

Endotoxin levels were subsequently depleted using the Nortek Endotoxin removal kit, after which the endotoxin levels were re-measured and found to be significantly lower (<0.1 EU/ml at the concentrations used in experiments).

To test the effect of conjugate C on HLA-A2 allo-proliferative responses MLRs were performed using PBMCs from HLA-A2 negative donors (used as responders) and HLA-A2 positive donors (used as stimulators) in the presence or absence of conjugate C. Addition of conjugate C resulted in a reduction of allo-proliferative responses.

Repeating this MLR using conjugate C where endotoxins it had been cleaned of endotoxin led to a reduction in allo-proliferative responses as measured by ³H-thymidine uptake on Day 7.

An HLA-A2 positive EBV transformed B-LCL cell line (C073) was used in MLR experiments and cell-mediated cytotoxicity experiments.

In the MLR 10⁵ PBMCs were added to a well in a 96-well U bottom plate and incubated with C073 cells (treated with mitomycin-C) in a ratio of 1:1, 1:3, 1:10 and 1:30 PBMCs:C073. A good proliferative response at 7 days was measured using ³H-thymidine uptake.

### Example 5—Binding Studies

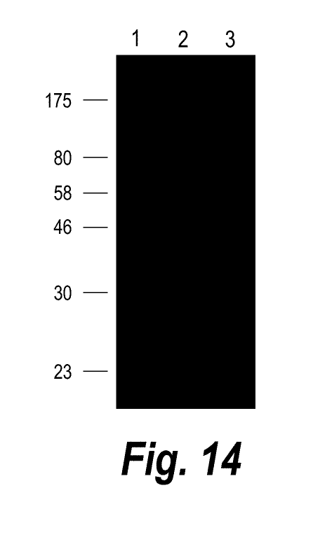

Immunoprecipitation (IP) experiments using HLA-A2 coupled to streptavidin agarose beads did not demonstrate binding of the conjugate A construct to HLA-A2. By contrast, conjugate B did show binding to HLA-A2 (FIG. 14).

Protocol for Immunoprecipitation Studies

30 μl of streptavidin agarose beads (Sigma Aldrich) were washed with 1 ml PBS. After washing, the streptavidin agarose beads were incubated with 1 μg biotinylated HLA-A2 (purchased from the Frank Hutchinson Cancer Research Centre) in 250 μl PBS for 1 hour to allow binding of biotinylated HLA-A2 to the beads. The beads were then incubated for 3 hours with 250 μl of supernatant from cells transfected with conjugate A or conjugate B. Beads were then washed 3 times with 1 ml PBS, prior to separation by SDS-PAGE and Western blot analysis.

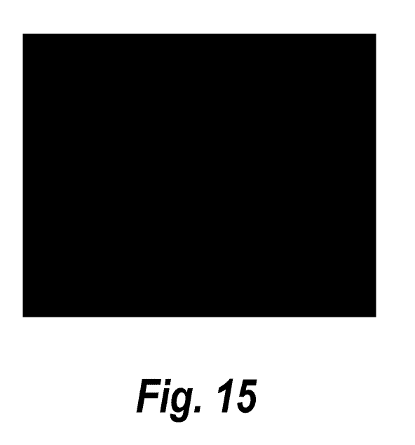

Western blot analysis used anti-HLA-G (4H84) as the detection antibody and this indicated that the HLA-A2 beads had pulled down conjugate B but not conjugate A (FIG. 15).

IP Experiments Using Various Anti-HLA-G mAbs to Assay the Conformation of HLA-G/β2 microglobulin or Conjugate B

30 μl protein G sepharose beads were washed once with 1 ml PBS then incubated with 1 μg of mAb in 250 μl PBS for 1 hour to allow binding. mAbs included the conformational mAbs 87G, MEM-G9 and W6/32 and the non-conformational mAb 4H84. Coupled beads were washed twice with PBS then incubated with either 250 μl PBS or supernatant taken from HLA-G/β2 microglobulin or conjugate B co-transfected COS7 cells for 3 hours. The beads were then washed 3 times with 1 ml PBS, prior to separation by SDS-PAGE and Western blotting.

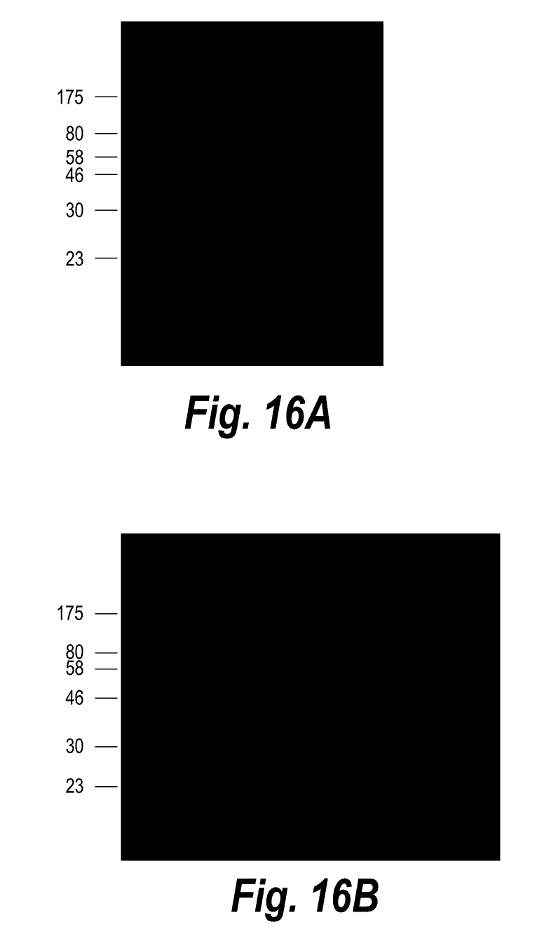

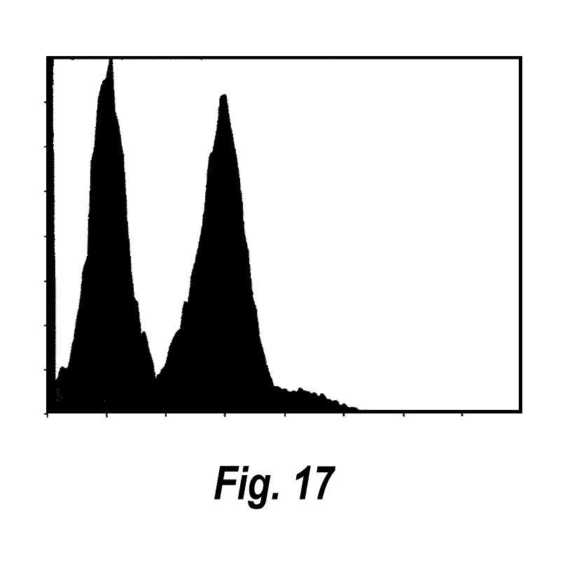

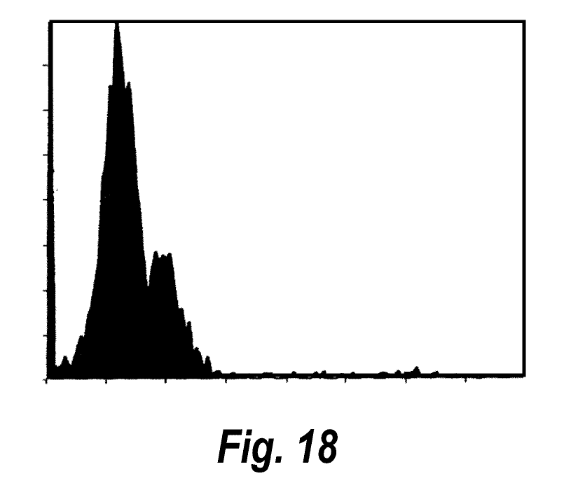

Western blot analysis revealed that the conformational antibodies were able to pull down HLA-G/β2M but not conjugate B suggesting that there might be folding problems with the HLA-G portion (FIGS. 16A-16B). Consequently, we have now recloned the G-body (original and B11 version) without the 132M portion of the molecule in the first instance.

FACS Analysis

FACS analysis was performed by incubating PBMCs with conjugate C for 1 hour on ice. Cells were then washed twice with PBS/1% BSA/0.1% azide. The secondary antibody (GAM FITC) was added and the cells were incubated for 1 hour on ice. After 2 further washes the cells were fixed in 500 μl BD CellFix prior to flow FACS analysis.

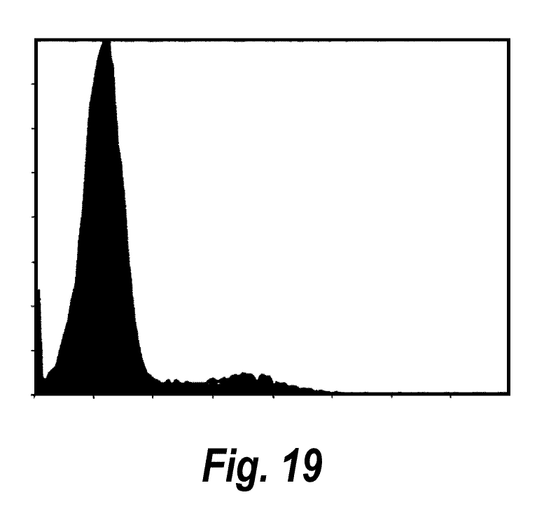

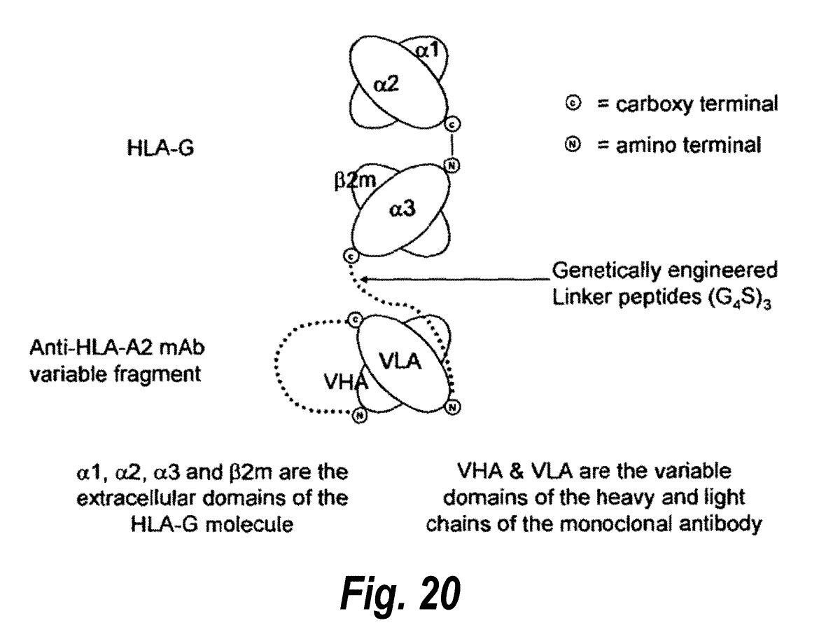

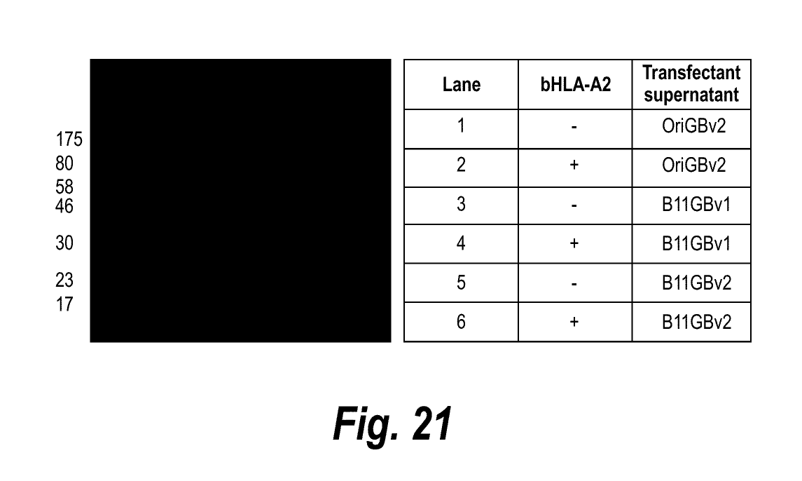

FACS analysis revealed that conjugate C was able to bind to HLA-A2 positive lymphocytes (FIG. 17) and HLA-A2 negative monocytes (FIG. 18) but not HLA-A2 negative lymphocytes (FIG. 19) with the detection antibody being goat-anti-mouse Ig.

### Example 5a—Re-Cloning of Original G-Body (ORIGBv1) and B11-Gbody (B11GBv1) without β2M Segment and Subsequent Binding Studies

ORIGBv1 and B11GBv1 were recloned without the β2m segment to determine if this would allow the HLA-G portion of the molecule to fold correctly.

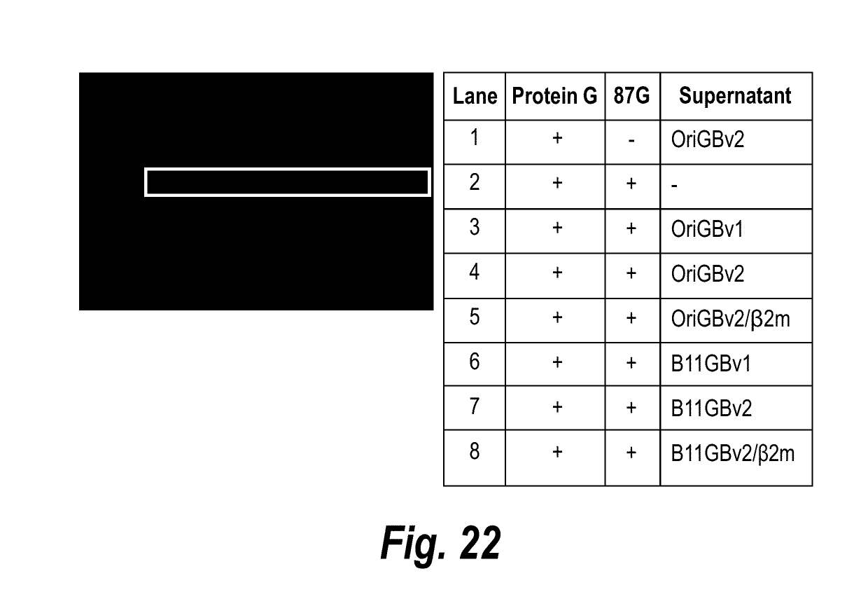

Further versions of the recombinant G-body without the β2m segment (and 6×His tag) “version 2” were generated by PCR cloning (FIG. 20). The ORIGBv1 and B11GBv1 plasmids were used as the template for PCR cloning. The new constructs were designed with 5′ NheI and 3′ BamHI restriction enzyme sites (underlined) to allow cloning into a pcDNA3.1(+) vector. The primers used for ORIGBv2 cloning were 5′-CTGGCTAGCACCACCATGGTGGTC and ATAGGATCCTCACGGGGGTGTCGTACGGGCTG-3′; whereas the primers used for B11GBv2 cloning were 5′-CTGGCTAGCACCACCATGGTGGTC and ATAGGATCCTCACCCGAGCACTGTCAGCTTGG-3′.

PCR products were run on an 0.8% agarose gel and bands corresponding to the expected size were extracted from the gel and incubated with NheI and BamHI restriction enzymes prior to being purified with the Qiagen PCR product purification kit.

pcDNA3.1(+) plasmid was also incubated with NheI and BamHI restriction enzyme prior to being run on an 0.8% agarose gel. The corresponding linearized plasmid was extracted from the gel. The linearized vector and RE digested inserts were ligated and transformed into TOP10 cells.

DNA sequencing confirmed that the ORIGBv1 and B11GBv1 plasmids had the intended sequence. Sequencing primers used were stock T7F and bGHR.

Binding Characteristics of ORIGBv2 and B11GBv2

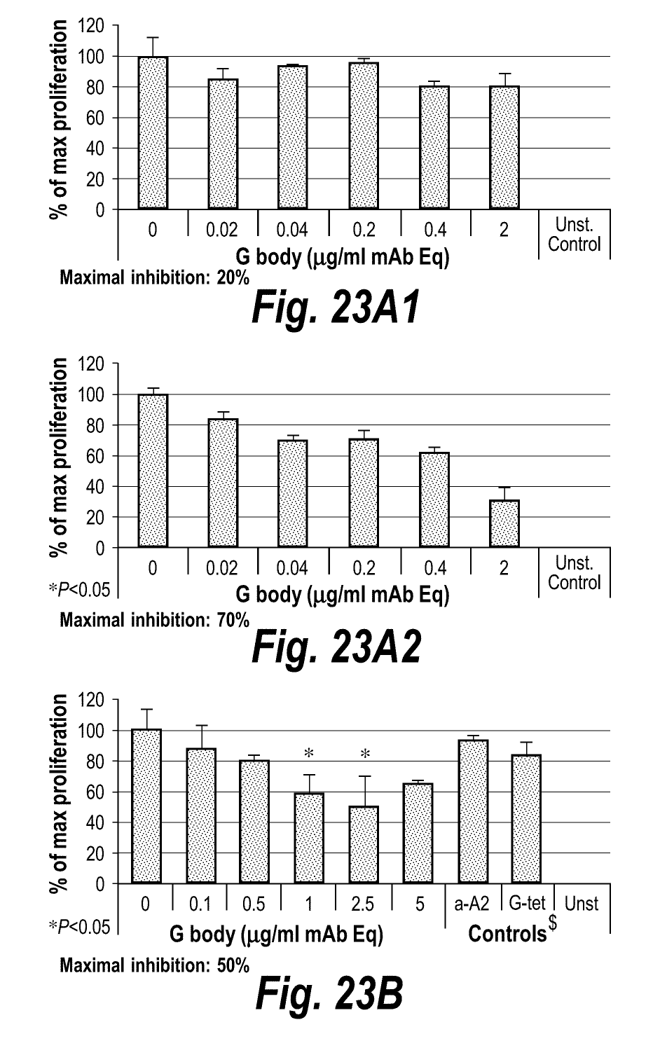

Immunoprecipitation experiments confirmed binding of B11GBv2 to HLA-A2 (FIG. 21). Of interest, ORIGBv2 also showed weak binding to HLA-A2.

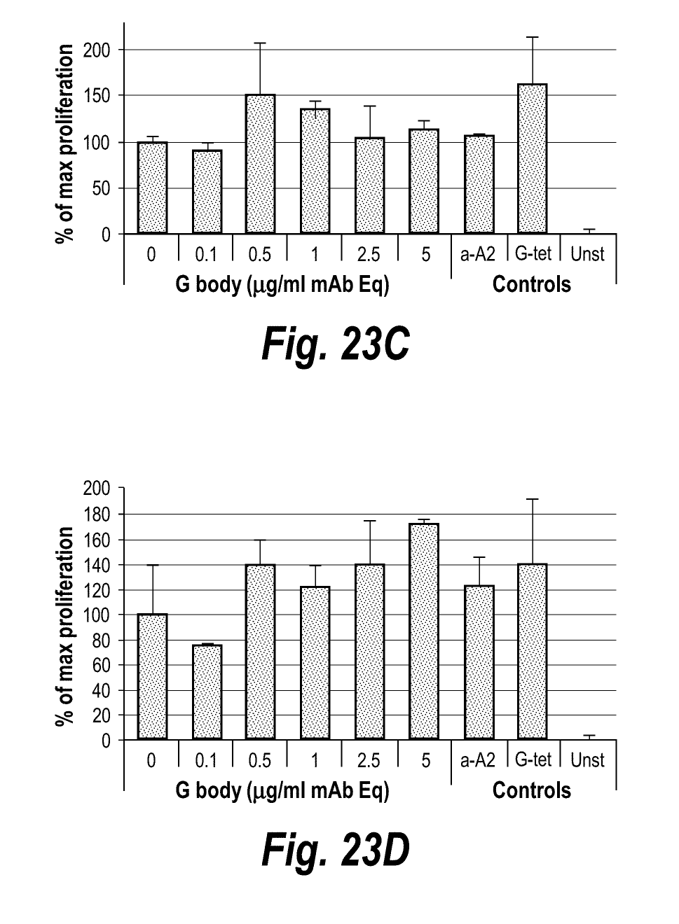

In addition, both ORIGBv2 and B11GBv2 showed greater binding to the conformational monoclonal antibodies 87G and MEM-G/9. FIG. 22 shows binding to 87G and the pattern is the same with MEM-G/9.

In summary, the B11GBv2 showed binding to HLA-A2 and had a HLA-G portion that had the correct conformational state. The ORIGBv2 had an HLA-G portion in the correct conformational state and probably had a degree of binding to HLA-A2 as well.

### Example 6—Effect of G-Body (Conjugate C as Defined in Example 1) on Proliferative Responses in PBMCs

Two different approaches were used to address the effect of G-Body on immune cell function and tested the role of G-Body on T-cell proliferation.

Reagents

Tissue culture medium used was RPMI 1640 (Gibco) supplemented with 10% FCS (heat inactivated for 30 min at 56° C., Sigma) 100 U/ml penicillin (Sigma) and 100 μg/ml streptomycin (Sigma) and 2 mM L-glutamine (Sigma). T cell activation was induced with Dynabeads Human T-activator CD3/CD28 (Invitrogen) at a bead:cell ratio of 1:5 or with the superantigen Staphylococcus aureus enterotoxin B (SEB, Sigma-Aldrich) used at a final concentration of 1 ng/ml.

Induction and Measurement of Proliferative Responses in PBMCs

PBMCs were isolated from buffy coats (National Blood Service, London) by density centrifugation on Lymphoprep (Nycomed), typed for HLA-A2 expression by flow cytometry (see below) and stored in liquid nitrogen in 90% FCS/10% DMSO until further use.

PBMC (1×10⁵ cells/250 μl) were cultured in 96-well round-bottom plates with increasing doses of conjugated G-Body (calculated as anti-HLA-A2 mAb Equivalent, range 0.1-2.5 μg/ml). In parallel, the following controls were also tested in some experiments: anti-HLA-A2 (2.5 μg/ml, IgG2b, clone BB7.2, grown in-house), IgG2b (2.5 μg/ml, eBioscience), HLA-G tetramers (2.5 μg/ml mAb Eq). PBMCs were activated with either SEB or anti-CD3/anti-CD28 coated beads. Proliferative responses were assessed by measuring the incorporation of [³H]-TdR (Amersham) by liquid scintillation spectroscopy after a pulse with 37 KBq/well during the last 16 h of a 3- and/or 7-day culture.

Evaluation of HLA-A2 Expression on PBMCs by Flow Cytometry

PBMCs were washed, resuspended and stained on ice for 30 min in PBS containing 1% BSA and 0.05% NaN₃. The cells were stained with FITC-conjugated mouse anti-human HLA-A2 mAb (IgG2b, 1 μg/1×10⁶ cells, clone BB7.2 in-house grown and conjugated) or the isotype-matched FITC-conjugated control mAb (MBL). Data were acquired on a Beckman Coulter flow cytometer and analysed using Beckman Coulter software.

Statistical Analysis

Data were analysed statistically with SPSS software using ANOVA with post-hoc Bonferroni tests. A P value of <0.05 was considered as the level of significance.

TABLE 2
Effect of G-Body on the proliferation of PBMCs

| | HLA-A2 | | G-Body inhibitory effect | |
|---|---|---|---|---|
| Stimulus | expression | Donor | Day 3 | Day 7 |
| CD3/CD28 | Positive | C | No effect | Max 70% at |
| beads | | | | 2 ug/ml |
| | | J | Max 50% at | No effect |
| | | | 2.5 ug/ml | |
| | | D | No effect | Max 50% at |
| | | | | 2.5 ug/ml |
| | | G | Max 40% at | Max 50% at |
| | | | 2.5 ug/ml | 2.5 ug/ml |
| | | L | No effect | No effect |
| | Negative | I | ND | No effect |
| | | K | ND | No effect |
| | | A | No effect | No effect |
| | | B | No effect | No effect |
| | | F | No effect | No effect |
| SEB | Positive | J | Max 70% at | Max 70% at |
| | | | 5 ug/ml | 5 ug/ml |
| | | C | Max 60% at | ND |
| | | | 5 ug/ml | |
| | | D | No effect | ND |
| | | G | No effect | ND |
| | | L | No effect | ND |
| | Negative | K | No effect | ND |
| | | A | No effect | ND |
| | | B | No effect | ND |
| | | F | No effect | ND |

ND: not done

G-Body Inhibits CD3/CD28 Induced PBMC Proliferation in a Dose Dependent Manner

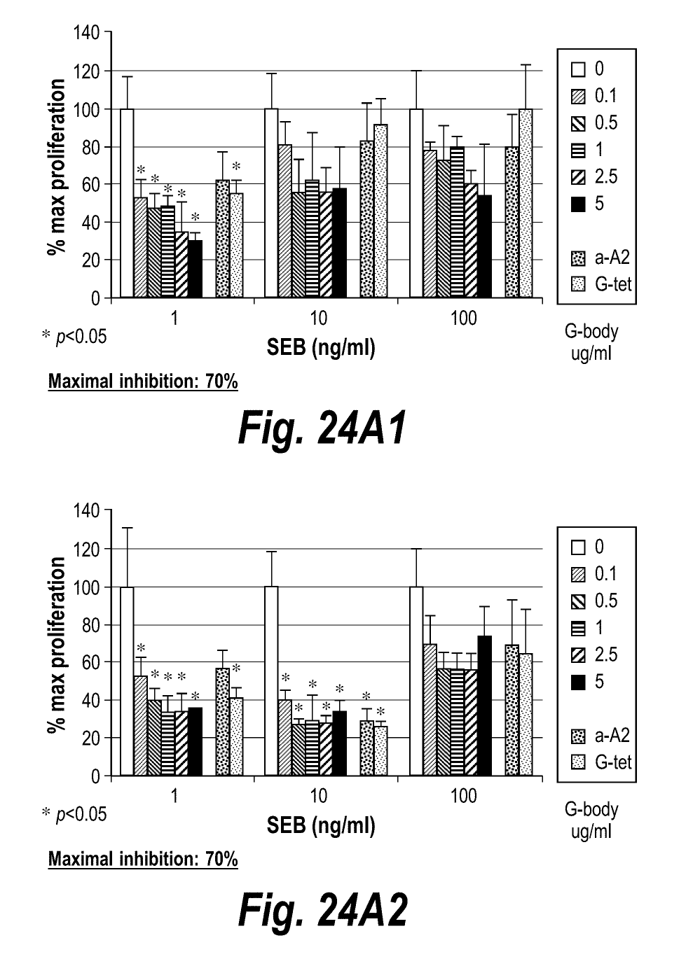

To address the question whether G-Body can modulate the proliferation of PBMC, cells were isolated from the peripheral blood of HLA-A2 positive or HLA-A2 negative donors (data not shown). Subsequently, PBMCs were polyclonally activated with beads coated with anti-CD3/anti-CD28 antibodies in the absence or presence of increasing doses of G-Body. Proliferation was evaluated after 3 and 7 days of culture. Using PBMC from an HLA-A2 positive donor (donor C), we observed a dose-dependent inhibition of proliferation reaching a significant 70% reduction in the incorporation of [³H]-TdR at the highest G-Body dose tested (FIG. 23A2). We subsequently tested more HLA-A2 positive donors and observed that (with e.g. donor J) a significant reduction of 50% in proliferation is already visible at 3 days post-activation (FIG. 23B). These data indicate that different donors respond to the inhibitory effects of G-Body with different kinetics. Furthermore, although it has been reported that targeting HLA-A2 (with anti-HLA-A2) or LILRs (with HLA-G tetramers) may result in immune suppression, it is striking to see that in our experiments there is a synergistic inhibitory effect when anti-HLA-A2 and HLA-G are combined in the form of G-Body. This is particularly relevant, because even when there is little or small inhibitory effect by the individual components (i.e. anti-HLA-A2 or HLA-G tetramers, FIG. 23B) on PBMC proliferation, G-Body still exhibits a strong inhibitory effect.

Interestingly, however, HLA-A2 negative PBMCs were not affected by G-Body (FIG. 23C, Table 2). HLA-A2 expression seems to be a pre-requisite for the inhibitory effect to take place as demonstrated by experiments described above.

G-Body is a Potent Inhibitor of Superantigen-Induced PBMC Proliferation

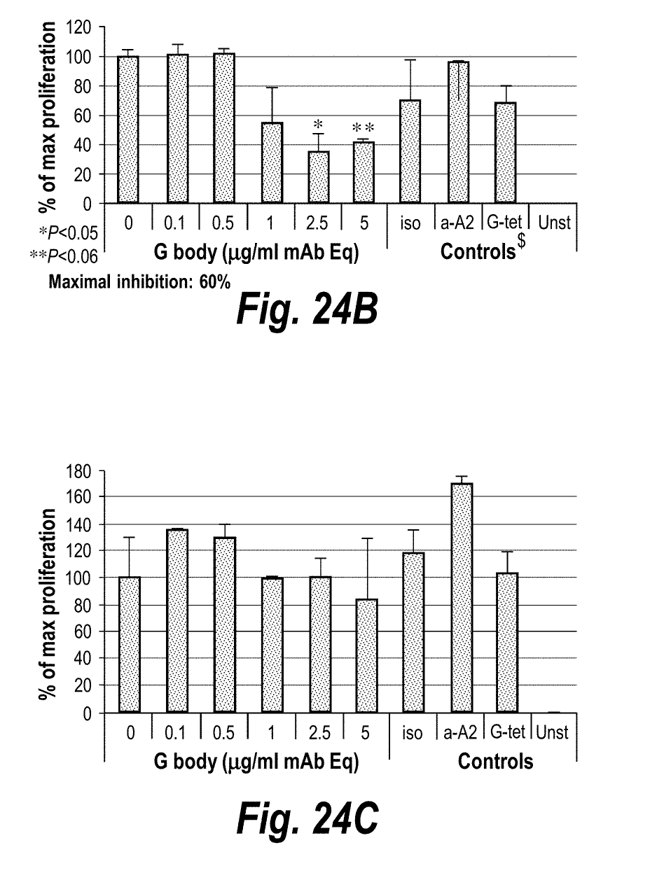

To evaluate the effect of G-Body during APC-T cell interactions, PBMC were activated oligoclonally with the MHC II/TCR crosslinker, superantigen SEB in the presence of increasing doses of G-Body. Initially, we tested the effect of G-Body in cultures activated with 3 different doses of SEB. At day 3 post-activation we observed a significant reduction of proliferation of up to 70% that was most obvious when SEB was used at the low dose of 1 ng/ml (FIG. 24A1). This effect also became obvious with 10 ng/ml SEB at day 7 post-activation, whereas no significance was reached with 100 ng/ml of SEB at either time point (FIG. 24A2).

Again, as observed in the experiments with CD3/CD28 beads, we observed a stronger effect of G-Body as compared to equimolar concentrations of its individual components (FIGS. 24A and 24B).

Finally, HLA-A2 negative PBMCs were not affected by G-Body (FIG. 24C, Table 2) and thus, HLA-A2 expression seems to be indispensable for the inhibitory effect.

We have shown a synergistic effect of HLA-G tetramers and anti-HLA-A2 when combined in the form of G-Body. This is a novel concept and supports the concept of the present invention.

Furthermore, HLA-A2 expression by immune cells seems to be a pre-requisite for the inhibitory effect to take place as demonstrated by experiments performed with HLA-A2 positive as opposed to HLA-A2 negative donors. Thus, these data support the concept that immune responses to antigens other than HLA-A2 would not be unduly compromised and confirms that the effect of G-Body would be specific to the HLA mismatch in a transplantation setting and therefore would be unlikely to cause blanket immunosuppression. The specificity to HLA-A2 in these results also supports the workings of the invention described herein.

Finally, our data demonstrate that the inhibitory effect of G-Body on PBMC proliferation is not due to any putative general toxicity of a foreign protein because there has been no significant inhibitory effect on the proliferation of HLA-A2 negative PBMCs.

### Example 7—Proposed Additional Experiments

Further experiments may be performed to further assess the effect of the conjugates upon immune responses.

Proposal 1

The inventors propose experiments using CFSE-painted K562 cells as targets in PBMC natural killer cell cytotoxicity experiments.

Proposal 2

Experiments are also proposed which test the effect of the conjugate on indirect sensitisation using monocyte-derived dendritic cells pulsed with a source of HLA-A2 as antigen presenting cells and autologous PBMCs as the responder cell population.

Proposal 3

The inventors propose using monocyte-derived dendritic cells pulsed with heat-shock treated K562-HLA-A2 transfectants to sensitize autologous PBMCs for 7 days and then test whether the addition of the conjugate has an effect when added at the sensitisation stage. The sensitized PBMCs would then be tested for their proliferative and cytotoxic response to HLA-A2.

Proposal 4

An extension of this work would be to target other common HLA mismatches; both class I and class II. For example, HLA-DR or HLA-B-specific G-body could have a beneficial effect if these molecules are mismatched between donor and recipient.

### Example 8—Materials and Methods

Procurement of HLA-A24 and HLA-B8 pcDNA3.1 Clones

HLA-A24 and HLA-B8 pcDNA3.1 plasmids were obtained from a collaborator after having been cloned from cDNA derived from EBV transformed B-LCL lines homozygous for the respective allele. E. coli were transformed with these plasmids followed by plasmid minipreparation.

Cloning of ILT2, ILT4 for Use in Ligand Binding Experiments

ILT2 and ILT4 receptors were cloned by PCR from pcDNA extracted from human peripheral blood mononuclear cells. Two-stage PCR with nested primers was used to incorporate HindIII and XhoI restriction sites at either ends of the PCR product. The PCR products were then inserted into pcDNA3.1 using the same process as described for soluble HLA-G but with the exception of using HindIII and XhoI restriction enzymes.

Procurement and Maintenance of Cell Lines

COS7, KG1, 293T, BB7.2 cell lines were procured from ATCC.

K562, JEG3, CO73 cell lines were procured from HPA Cultures.

W6/32, ZM3.8, 42D1, U937 were obtained from collaborators.

Base Medium

The base medium for all cell lines was supplemented with 10% FCS, L-glutamine and penicillin/streptomycin, apart from Eagle's Minimal Essential Medium (EMEM) which was also supplemented with non-essential amino acids and VPSFM (serum-free media) which had no FCS added.

COS7 Cells

These adherent cells were maintained in Dulbecco's Modified Eagle Medium (DMEM) prior to being adapted to VPSFM for serum free medium culture. The cells were detached with trypsin (which was changed to TrypLE after the switch to serum free medium) prior to splitting twice a week.

293T and JEG3

These adherent cells were maintained in DMEM. They were split twice a week after detachment with trypsin.

KG1

These suspension cells were maintained in Iscove's modified DMEM and were split twice a week.

K562, U937 and C073

These suspension cells were all maintained in RPMI and were split twice a week.

Transfected Cells Lines

K562-HLA-A2, K562-ILT2 and K562-ILT4 cell lines were maintained in RPMI supplemented with 700 μg/ml G418 (HLA-A2) or 800 μg/ml (ILT2 and ILT4) to maintain selective pressure on the cells. Cell lines were split twice a week.

Hybridomas and Monoclonal Antibody Extraction

All hybridomas were grown in DMEM and split twice a week.

Hybridomas were grown for 7 days prior to harvest of mAbs from the supernatant. mAbs were isolated using the MAbTrap kit (GE Life Sciences) which utilised a protein-G column for antibody binding followed by elution.

Transformation of Competent E. coli

Competent E. coli (TOP10; Invitrogen or NEB10beta; New England Biolabs) were transformed with plasmid DNA according to manufacturer's instructions. E. coli were thawed on ice, incubated with plasmid DNA for 30 minutes, heat shocked at 42° C. for 30 seconds then incubated on ice for a further 2 to 5 minutes. 250 μl or 950 μl SOC medium (for TOP10 and NEB10beta respectively) was then added to the E. coli, followed by incubation in a 37° C. shaking incubator for 60 minutes. Subsequently, 20 μl and 200 μl of the mixtures were plated on selective LB agar plates and incubated at 37° C. overnight.

Glycerol Stocks

Glycerol stocks of every clone were made by adding 800 μl of LB broth containing exponentially growing cells to 200 μl sterile glycerol. Glycerol stocks were stored at −80° C.

Peripheral Blood Mononuclear Cell Separation

Human peripheral blood mononuclear cells (PBMCs) were separated from whole blood or buffy coat by using Lymphoprep. Whole blood diluted 1:1 with PBS was layered on Lymphoprep and centrifuged at 800G for 20 minutes. The PBMC layer was then harvested and washed with Hank's Buffered Salt Solution (HBSS) before being used in experiments or frozen for storage.

DNA Minipreparations

DNA minipreparations were obtained by plating or streaking transformed TOP10 or NEB10 beta E. coli on LB agar plates containing selective medium (carbenicillin 100 μg/ml) and incubated at 37° C. overnight. Single colonies were then incubated in 6 ml LB broth containing carbenicillin and incubated at 37° C. overnight in a shaking incubator. 4 ml LB culture was then centrifuged and plasmid DNA was extracted from the pellet using the Qiagen Qiaprep Miniprep kit. The DNA was eluted in dH₂O, the concentration measured using a Nanodrop ND-1000 spectrophotometer followed by storage at −20° C.

DNA Midipreparations/Maxipreparations

DNA midipreparations/maxipreparations were obtained by plating or streaking transformed TOP10 or NEB10 beta E. coli on LB agar plates containing selective medium (carbenicillin 100 μg/ml) and incubating at 37° C. overnight. Single colonies were added to 5 ml LB broth containing carbenicillin and incubated for 8 hours at 37° C. in a shaking incubator. 1 or 5 ml LB culture was then added to 50 or 250 ml LB broth containing carbenicillin (for midi- and maxipreparations respectively) and incubated overnight at 37° C. in a shaking incubator. The LB broth was centrifuged and plasmid DNA was extracted from the resulting pellet using the Sigma GenElute HP Midiprep or Maxiprep kit. DNA was eluted in dH₂O, the concentration measured using a Nanodrop ND-1000 spectrophotometer followed by storage at −20° C.

Functional Experiments

All functional experiments were performed in RPMI medium containing 10% heat-inactivated FCS and penicillin/streptomycin.

Transfection of Cell Lines

Transient and stable transfections were performed using either the Superfect or Attractene transfection reagent from Qiagen in accordance with the manufacturer's instructions: Cells were seeded either 24 hours or immediately prior to tranfection. Plasmid DNA and the transfection reagent were mixed in the medium and incubated for 10 to 15 minutes at room temperature. This mixture was then added to the cells to be transfected. When the Superfect transfection reagent was used, the medium for cells was changed after 2-3 hours incubation.

Flow Cytometry (FACS) Experiments

Flow cytometry experiments were performed using a Beckmann Coulter Cytomics FC500 flow cytometer.

Painting with CFSE

Target cells used in cytotoxicity experiments were washed twice with PBS and resuspended at a concentration of 1-2×10⁶ cells/ml in PBS. Carboxyfluorescein succinimidyl ester (CFSE) was then added to the cells at a final concentration of 250 nM CFSE followed by 5 minute incubation in the dark. The cells were then washed three times with complete medium to neutralize any unbound CFSE prior to use.

Cell Mediated Cytotoxicity Experiments

CFSE-painted target cells were incubated with unpainted effector cells at ratios of 1:6, 1:12, 1:25 and 1:50 in 96 well U bottom plates. The plates were then centrifuged at 250G for 4 minutes followed by incubation at 37° C. for 4 hours. 7-amino-actinomycin D (7-AAD) was then added to the cells to a concentration of 10 μg/ml followed by 10 to 15 minute incubation in the dark. Cells were then transferred to a FACS tube followed by FACS analysis.

DNA Sequencing

DNA sequencing was performed using Applied Biosystems 3730 DNA analyzers by Geneservice. Standard sequencing primers or custom designed primers were occasionally provided by the company.

DNA Agarose Gel Electrophoresis

Agarose gels for use in DNA electrophoresis were formed by dissolving agarose in 0.5× Tris-borate-EDTA buffer. 0.5× Tris-borate-EDTA buffer was also used as an electrophoresis running buffer.

DNA Gel Extraction

All DNA extractions from agarose gels were performed using the Qiagen Qiaquick Gel Extraction kit in accordance with the manufacturer's instructions and the DNA was eluted with dH₂O.

DNA Ligation

All DNA ligations were performed using T4 DNA ligase and T4 DNA ligase buffer (New England Biolabs). Ligation reactions were incubated overnight at 16° C.

RNA Extraction

Total RNA extraction from either PBMCs or cell lines was performed using the Qiagen RNEasy Mini extraction kit.

Reverse Transcription

Reverse transcription of mRNA into cDNA was performed using the Qiagen Omniscript Reverse transcription reagent. Template RNA was incubated at 37° C. for 60 minutes with Omniscript RT, dNTPs, anchored oligo-dT primers and RNase inhibitor according to the manufacturer's instructions. Reaction products were subsequently used in PCR reactions or stored at −20° C.

Polymerase Chain Reaction

Polymerase chain reactions (PCR) were performed using either the Pwo polymerase kit (Roche) or the Phusion polymerase kit (New England Biolabs), both of which are engineered for high fidelity. Proprietary buffers and reagents were supplied with the kits, and the manufacturer's recommendations for their use were followed.

SDS-PAGE

SDS-PAGE experiments were performed using the Laemmli buffer system with discontinuous polyacrylamide gels and Tris-glycine-SDS buffer was used for SDS-PAGE experiments. Gels were cast using Bio-Rad Mini Protean gel casting stands. Samples were run using the Bio-Rad Mini-Protean Tetra cell. Laemmli reducing buffer was added to the samples then incubated at 95-100° C. before loading on to the gels for electrophoresis.

Western Blotting

Protein was transferred from SDS-PAGE gels to PVDF membranes (Hybond-P, GE Life Sciences) using the Bio-Rad Trans-Blot SD Semi-Dry Electrophoretic Transfer Cell in the presence of Bjerrum and Schafer-Nielsen transfer buffer (48 mM Tris. 39 mM glycine, 20% methanol). Transfer times employed were in accordance to the manufacturer's recommendations.

Membranes were then incubated in TBS-T (Tris buffered saline-Tween) buffer with 5% skimmed milk for 60 minutes. Membranes were then washed, followed by overnight incubation at 4° C. with the primary antibody in TBS-T/2.5% BSA. The membranes were then washed twice with TBS-T and incubated with the secondary antibody (goat anti-mouse HRP) in TBS-T for 60 minutes at room temperature. The membrane was then washed a further 3 times.

ECL reagent (GE Life Sciences) was then added to the membrane for 1 minute before the membrane was exposed to autoradiography film (Hyperfilm ECL, GE Life Sciences). Exposure times ranged between 2 to 60 minutes to obtain the best quality images and the film was developed in an automated film processor.

### SEQUENCE LISTING

(Reference HLA-G with leader peptide)
SEQ ID NO: 1
MVVMAPRTLFLLLSGALTLTETWAGSHSMRYFSAAVSRPGRGEPRFI
AMGYVDDTQFVRFDSDSACPRMEPRAPWVEQEGPEYWEEETRNTKAH
AQTDRMNLQTLRGYYNQSEASSHTLQWMIGCDLGSDGRLLRGYEQYA
YDGKDYLALNEDLRSWTAADTAAQISKRKCEAANVAEQRRAYLEGTC
VEWLHRYLENGKEMLQRADPPKTHVTHHPVFDYEATLRCWALGFYPA
EIILTWQRDGEDQTQDVELVETRPAGDGTFQKWAAVVVPSGEEQRYT
CHVQHEGLPEPLMLRWKQSSLPTIPIMGIVAGLVVLAAVVTGAAVAA
VLWRKKSSD
(HLA-G sequence, along with the leader peptide
and with a truncated transmembrane/cytoplasmic
domain for synthesis of the recombinant
conjugate)
SEQ ID NO: 2
MVVMAPRTLFLLLSGALTLTETWAGSHSMRYFSAAVSRPGRGEPRFI
AMGYVDDTQFVRFDSDSACPRMEPRAPWVEQEGPEYWEEETRNTKAH
AQTDRMNLQTLRGYYNQSEASSHTLQWMIGCDLGSDGRLLRGYEQYA
YDGKDYLALNEDLRSWTAADTAAQISKRKCEAANVAEQRRAYLEGTC
VEWLHRYLENGKEMLQRADPPKTHVTHHPVFDYEATLRCWALGFYPA
EIILTWQRDGEDQTQDVELVETRPAGDGTFQKWAAVVVPSGEEQRYT
CHVQHEGLPEPLMLRWKQSSLPTIPS
(Example of a conjugate sequence)
SEQ ID NO: 3
MVVMAPRTLFLLLSGALTLTETWAGSHSMRYFSAAVSRPGRGEPRFI
AMGYVDDTQFVRFDSDSACPRMEPRAPWVEQEGPEYWEEETRNTKAH
AQTDRMNLQTLRGYYNQSEASSHTLQWMIGCDLGSDGRLLRGYEQYA
YDGKDYLALNEDLRSWTAADTAAQISKRKCEAANVAEQRRAYLEGTC
VEWLHRYLENGKEMLQRADPPKTHVTHHPVFDYEATLRCWALGFYPA
EIILTWQRDGEDQTQDVELVETRPAGDGTFQKWAAVVVPSGEEQRYT
CHVQHEGLPEPLMLRWKQSSLPTIPSGGGGSGGGGSGGGGSDVLMTQ
TPLSLPVSLGDQVSISCRSSQSIVHSNGNTYLEWYLQKPGQSPKLLI
YKVSNRFSGVPDRFSGSGSGTDFTLKISRVEAEDLGVYYCFQGSHVP
RTFGGGTKLEIKRADAAEQKLISEEDLHHHRARYKRQVQLQQSGPEL
VKPGASVKMSCKASGYTFTSYHIQWVKQRPGQGLEWIGWIYPGDGST
QYNEKFKGKTTLTADKSSSTAYMLLSSLTSEDSAIYFCAREGTYYAM
DYWGQGTSVTVSSARTTPPGGGGSIQRTPKIQVYSRHPAENGKSNFL
NCYVSGFHPSDIEVDLLKNGERIEKVEHSDLSFSKDWSFYLLYYTEF
TPTEKDEYACRVNHVTLSQPKIVKWDRDMTGHHHHHH
(Example of B11-based conjugate sequence)
SEQ ID NO: 4
MVVMAPRTLFLLLSGALTLTETWAGSHSMRYFSAAVSRPGRGEPRFI
AMGYVDDTQFVRFDSDSACPRMEPRAPWVEQEGPEYWEEETRNTKAH
AQTDRMNLQTLRGYYNQSEASSHTLQWMIGCDLGSDGRLLRGYEQYA
YDGKDYLALNEDLRSWTAADTAAQISKRKCEAANVAEQRRAYLEGTC
VEWLHRYLENGKEMLQRADPPKTHVTHHPVFDYEATLRCWALGFYPA
EIILTWQRDGEDQTQDVELVETRPAGDGTFQKWAAVVVPSGEEQRYT
CHVQHEGLPEPLMLRWKQSSLPTIPSGGGGSGGGGSGGGGSQVQLVQ
SGGGVVQPGGSLRVSCAASGVTLSDYGMHWVRQAPGKGLEWMAFIRN
DGSDKYYADSVKGRFTISRDNSKKTVSLQMSSLRAEDTAVYYCAKNG
ESGPLDYWYFDLWGRGTLVTVSSGGGGSGGGGSGGGGSDVVMTQSPS
SLSASVGDRVTITCQASQDISNYLNWYQQKPGKAPKWYDASNLETGV
PSRFSGSGSGTDFTFTISSLQPEDIATYYCQQYDNLPPTFGGGTKLT
VLGGGGGSGGGGSGGGGSIQRTPKIQVYSRHPAENGKSNFLNCYVSG
FHPSDIEVDLLKNGERIEKVEHSDLSFSKDWSFYLLYYTEFTPTEKD
EYACRVNHVTLSQPKIVKWDRDMTGHHHHHH

1. A conjugate comprising a first portion connected to a second portion, wherein the first portion binds to an MHC Class I molecule and the second portion has HLA-G activity.

2. The conjugate according to claim 1, wherein the first portion comprises an antibody or antibody fragment.

3. The conjugate according to claim 2, wherein the antibody is a monoclonal antibody.

4. The conjugate according to claim 2, wherein the antibody is an antigen binding immunoglobulin fragment.

5. The conjugate according to claim 2,
wherein the monoclonal antibody is derived from hybridoma BB7.2 or the clone B11.

6. A conjugate according to claim 1 wherein the first portion comprises an aptamer.

7. The conjugate according to claim 1, wherein the first portion and second portion are connected by a linker.

8. The conjugate according to claim 7, wherein the linker is a peptide.

9. The conjugate according to claim 1, wherein the second portion comprises HLA-G or a fragment thereof.

10. The conjugate according to claim 9, wherein the second portion comprises a polypeptide amino acid sequence selected from the group consisting of SEQ ID NO: 1 and 2.

11. The conjugate according to claim 1, wherein the first portion and second portion are connected by a covalent or non-covalent binding interaction.

12. A conjugate according to claim 11, wherein the connection between the first portion and the second portion is via a binding interaction mediated by biotin and streptavidin.

13. A conjugate according to claim 12, wherein the first portion is linked to biotin and the second portion is linked to streptavidin.

14. A conjugate according to claim 13, wherein the variable domains of the heavy and light chain of an anti-HLA antibody are linked to streptavidin and the extracellular domain of HLA-G is linked to biotin.

15. A pharmaceutical composition comprising a conjugate according to claim 1.

16. A method of preventing graft rejection in a transplant patient comprising administering a conjugate according to claim 1 to the patient before, during and/or after transplant surgery.

17. A method of inducing tolerance to a graft in a transplant patient comprising administering a conjugate according to claim 1 to the patient before, during and/or after transplant surgery.

18. A method of preventing graft rejection in a transplant patient comprising administering a pharmaceutical composition according to claim 15 to the patient before, during and/or after transplant surgery.

19. A method of inducing tolerance to a graft in a transplant patient comprising administering a pharmaceutical composition according to claim 15 to the patient before, during and/or after transplant surgery.
